(19) United States
(12) Patent Application Publication
(10) Pub. No.: US 2005/0256410 A1
(43) Pub. Date: Nov. 17, 2005

(54) Apparatus and method for an ultrasonic probe capable of bending with aid of a balloon

(75) Inventors: Robert A. Rabiner, North Reading, MA (US); Bradley A. Hare, Chelmsford, MA (US); Rebecca I. Marciante, North Reading, MA (US); Mark J. Varady, Andover, MA (US); Daniel E. Rabiner, North Reading, MA (US)

Correspondence Address: FISH & RICHARDSON PC, P.O. BOX 1022, MINNEAPOLIS, MN, 55440-1022, US

(73) Assignee: OmniSonics Medical Technologies, Inc.

(21) Appl. No.: 10/846,038

(22) Filed: May 14, 2004

(51) Int. Cl. A61B 17/20; A61B 8/14

(52) U.S. Cl. 600/470; 600/462; 600/466; 600/467

(57) ABSTRACT

The present invention provides an apparatus and a method for an ultrasonic probe capable of bending, flexing and deflecting with the aid of a balloon to remove a biological material. An ultrasonic medical device includes a balloon catheter, a balloon that is supported by the balloon catheter, an inflation lumen located along a longitudinal axis of the balloon catheter and an ultrasonic probe located along an outside surface of the balloon catheter wherein the ultrasonic probe engages an outer surface of the balloon. The ultrasonic probe is inserted through at least one engaging mechanism on an outside surface of the balloon catheter. The inflated balloon causes the ultrasonic probe to bend, allowing the ultrasonic probe to move along a bend in a vasculature and increase a surface area of the ultrasonic probe in communication with the biological material for ablation.

### RELATED APPLICATIONS

None.

### FIELD OF THE INVENTION

The present invention relates to an ultrasonic medical device, and more particularly to an apparatus and a method for an ultrasonic probe capable of bending flexing and deflecting with the aid of a balloon to ablate a biological material.

### BACKGROUND OF THE INVENTION

Vascular occlusive disease affects millions of individuals worldwide and is characterized by a dangerous blockage of blood vessels. Vascular occlusive disease includes thrombosed hemodialysis grafts, peripheral artery disease, deep vein thrombosis, coronary artery disease and stroke. Vascular occlusions (including, but not limited to, clots, intravascular blood clots or thrombus, occlusional deposits, such as calcium deposits, fatty deposits, atherosclerotic plaque, cholesterol buildup, fibrous material buildup and arterial stenoses) result in the restriction or blockage of blood flow in the vessels in which they occur. Occlusions result in oxygen deprivation (“ischemia”) of tissues supplied by these blood vessels. Prolonged ischemia results in permanent damage of tissues which can lead to myocardial infarction, stroke, or death. Targets for occlusion include coronary arteries, peripheral arteries and other blood vessels.

The disruption of an occlusion can be affected by pharmacological agents, mechanical methods, ultrasonic methods or combinations of all three. Many procedures involve inserting a medical device into a vasculature of the body. Medical devices include, but are not limited to, probes, catheters, wires, tubes and similar devices. In some cases, the medical device delivers a pharmacological agent to the site of the occlusion.

Navigation of a probe within a vasculature of a body to a site of an occlusion can be a challenging process for a medical professional. The difficulty of the navigation lies in the type of vasculature that is being navigated, the path of the particular vasculature that is being navigated, the degree of blockage of the occlusion and the physical properties of the probe. Many occlusions reside at locations in the vasculature that are difficult to reach. Probes need to have a degree of rigidity to control the insertion process through the tortuous paths of the vasculature. Often, a torque is applied to the probe to move the probe through the vasculature. The probe must have sufficient rigidity to withstand the applied forces and torques when attempting to move the probe to the occlusion site within the vasculature. In addition, probes need to have a degree of flexibility so the probe can flex, bend and curve according to the path of the vasculature. The flexibility reduces the potential risk of damage to the vasculature as the probe is being navigated within the vasculature.

U.S. Pat. No. 5,902,289 to Swartz et al. discloses a precurved guiding introducer system and a process for treatment of atrial arrhythmia. The Swartz et al. device provides five different guiding introducers for procedures within the left atrium and four shaped guiding introducers for proceeding within the right atrium. The Swartz et al. device is specific to the left and right atrium and could not be used in other vasculatures having an occlusion. The Swartz et al. device has a precurved guiding introducer system that limits the effectiveness of energy transfer to the occlusive material and could not treat occlusions at bends and downstream of bends in the vasculature.

U.S. Pat. No. 4,732,152 to Wallsten et al. discloses a device and method for implantation of a prosthesis in areas that are difficult to access by positioning an inflatable balloon ahead or behind a double walled section containing the prosthesis to widen the lumen. The Wallsten et al. device includes a hose surrounding a probe that is moved to the site where the prosthesis is to be delivered and the prosthesis is implanted. The Wallsten hose is bulky and could not be used in varying vasculatures. In addition, the Wallsten hose would limit the effectiveness of energy transfer through the hose and could not be used in an application where a medical device would ablate an occlusive material in a vasculature of the body. The inflatable balloon that is positioned ahead or behind the double walled section is solely used to open up the lumen and could not be used to guide the Wallsten et al. device.

U.S. Pat. No. 5,531,664 to Adachi et al. discloses a bending actuator having a coil sheath with a fixed distal end and a free proximal end. The Adachi et al. device is complex and comprises a coil sheath, a plurality of wires, a plurality of valves, control circuits and many other parts that make the device bulky. The Adachi et al. device comprises a complicated mechanism of providing for a probe device that can be set into any desired bent condition. In addition, the Adachi et al. device would not be effective for the transmission of energy to a site of an occlusion and the size of the Adachi et al. device would limit its use in many vasculatures.

The prior art devices and methods for bending, flexing and deflecting a probe in the vasculature of the body to ablate occlusions are complex, ineffective and complicated. The prior art devices do not provide effective treatment of occlusions at the bend in the vasculature and further downstream of the bend. The prior art devices are complex and require large components to be inserted into a vasculature of the body that can harm the vasculature. The prior art devices have components that limit the effectiveness of the device in being able to ablate an occlusion. Therefore, there is a need in the art for an apparatus and method for bending an ultrasonic probe within the vasculature in the body to ablate occlusions that allows for effective energy transfer to ablate the occlusions, can be used in varying vasculatures, does not compromise the functionality of the probe and does not adversely affect the vasculature or the patient.

### SUMMARY OF THE INVENTION

The present invention is an apparatus and a method for an ultrasonic probe capable of bending, flexing and deflecting with the aid of a balloon to ablate a biological material. The present invention is an ultrasonic medical device comprising a balloon catheter having a proximal end, a distal end and a longitudinal axis therebetween and a balloon supported by the balloon catheter. The ultrasonic medical device includes an ultrasonic probe located along an outside surface of the balloon catheter, the ultrasonic probe engaging an outer surface of the inflated balloon. The ultrasonic medical device includes an inflation lumen located along the longitudinal axis of the balloon catheter, with an inner surface of the balloon in communication with the inflation lumen.

The present invention is an ultrasonic medical device comprising a balloon catheter comprising at least one engaging mechanism located along an outside surface of the balloon catheter. The ultrasonic medical device includes a balloon that engages the outside surface of the balloon catheter, the balloon having an outer surface, an inner surface, a proximal end and a distal end. An elongated, ultrasonic probe located along a longitudinal axis of the balloon catheter extends through at least one engaging mechanism and engages an outer surface of the inflated balloon. An inflation lumen located along the longitudinal axis of the balloon catheter is in communication with the balloon.

The present invention is a balloon catheter comprising a proximal end, a distal end and a longitudinal axis therebetween. The balloon catheter comprises an inflation lumen located along the longitudinal axis of the balloon catheter and a balloon supported by the balloon catheter, an inner surface of the balloon in communication with the inflation lumen. The balloon catheter comprises a distal engaging mechanism extending from an outside surface of the distal end of the balloon catheter.

The present invention is a balloon catheter comprising a proximal end, a distal end and a longitudinal axis therebetween. The balloon catheter comprises an inflation lumen located along the longitudinal axis of the balloon catheter and a balloon supported by the balloon catheter, an inner surface of the balloon in communication with the inflation lumen. The balloon catheter comprises a channel along an outside surface of the balloon catheter.

The present invention is an ultrasonic probe comprising a proximal end, a distal end and a longitudinal axis therebetween. The ultrasonic probe comprises a proximal section located proximal to the distal end and a flexible section located between the distal end and the proximal section. The flexible section comprises a diameter smaller than both a diameter of the proximal section of the ultrasonic probe and a diameter of the distal end of the ultrasonic probe.

The present invention provides a method of moving an ultrasonic probe along a bend in a vasculature to ablate an occlusion in the vasculature. The ultrasonic probe is inserted through a proximal engaging mechanism located on an outside surface of a balloon catheter. The ultrasonic probe is moved over an outer surface of a balloon supported by the balloon catheter and through a distal engaging mechanism located on the outside surface of the balloon catheter. The balloon catheter is advanced until the balloon is adjacent to the bend in the vasculature. The balloon is inflated, causing the outer surface of the balloon to engage the ultrasonic probe and bend the ultrasonic probe between the proximal engaging mechanism and the distal engaging mechanism. The ultrasonic probe is advanced along the outer surface of the balloon to move the ultrasonic probe along the bend in the vasculature and position the ultrasonic probe proximal to the occlusion. The ultrasonic probe is energized to ablate the occlusion at the bend in the vasculature.

The present invention provides a method of moving a flexible ultrasonic probe that is capable of taking a non-linear shape along a bend within a vasculature of a body to remove a biological material. A balloon catheter having a balloon in communication with an outside surface of the balloon catheter and the flexible ultrasonic probe extending along an outer surface of the balloon are provided. The balloon is inflated and a surface area of the flexible ultrasonic probe in communication with the biological material is increased. The flexible ultrasonic probe is moved along the outer surface of the balloon to move the flexible ultrasonic probe along the bend in the vasculature toward the biological material. An ultrasonic energy source is activated to provide ultrasonic energy to the ultrasonic probe to remove the biological material.

The present invention is an ultrasonic medical device comprising an ultrasonic probe capable of bending with the aid of a balloon to ablate a biological material. The inflated balloon causes the ultrasonic probe to bend and increase a surface area of the ultrasonic probe in communication with the occlusion. The present invention provides an ultrasonic medical device that is simple, effective, safe, reliable and cost effective.

### BRIEF DESCRIPTION OF THE DRAWINGS

The present invention will be further explained with reference to the attached drawings, wherein like structures are referred to by like numerals throughout the several views. The drawings shown are not necessarily to scale, with emphasis instead generally being placed upon illustrating the principles of the present invention.

FIG. 1 is a side plan view of an ultrasonic medical device of the present invention including a balloon catheter that supports a balloon and an ultrasonic probe located outside of the balloon catheter.

FIG. 2A is a side plan view of an ultrasonic probe of the present invention capable of operating in a transverse mode.

FIG. 2B is a side plan view of an ultrasonic probe of the present invention having an approximately uniform diameter from a proximal end of the ultrasonic probe to a distal end of the ultrasonic probe.

FIG. 3 is a fragmentary side plan view of an ultrasonic medical device of the present invention including a balloon catheter that supports a balloon and an ultrasonic probe inserted into a proximal engaging mechanism and a distal engaging mechanism, wherein the ultrasonic medical device is located adjacent to a bend in a vasculature.

FIG. 4 is a longitudinal cross section view of an ultrasonic medical device of the present invention with the balloon uninflated, showing an ultrasonic probe inserted through a flat section of an opening of a proximal engaging mechanism and a flat section of an opening of a distal engaging mechanism.

FIG. 5 is a longitudinal cross section view of an ultrasonic medical device of the present invention with the balloon inflated, showing an ultrasonic probe deflected along a chamfered edge of a proximal engaging mechanism and a chamfered edge of a distal engaging mechanism.

FIG. 6A is an end view of an embodiment of a first face of a proximal engaging mechanism and a second face of a distal engaging mechanism of the present invention comprising a keyhole-shaped opening with an upper section located on top of a smaller lower section, an ultrasonic probe located in the upper section of the keyhole-shaped opening.

FIG. 6B is an end view of an embodiment of a second face of a proximal engaging mechanism and a first face of a distal engaging mechanism of the present invention comprising a keyhole-shaped opening with a smaller upper section located on top of a lower section, an ultrasonic probe located in a lower section of the keyhole-shaped opening.

FIG. 7A is an end view of an embodiment of a first face of a proximal engaging mechanism and a second face of a distal engaging mechanism of the present invention comprising a keyhole-shaped opening with an upper section located on top of a smaller lower section, an ultrasonic probe located in the smaller lower section of the keyhole-shaped opening.

FIG. 7 is an end view of an embodiment of a second face of a proximal engaging mechanism and a first face of a distal engaging mechanism of the present invention comprising a keyhole-shaped opening with a smaller upper section located on top of a lower section, an ultrasonic probe located in the smaller upper section of the keyhole-shaped opening.

FIG. 8 is a longitudinal cross section view of an embodiment of a proximal engaging mechanism and a distal engaging mechanism.

FIG. 9 is fragmentary side plan views of an alternative embodiment of an ultrasonic medical device of the present invention including a balloon catheter that supports a balloon and an ultrasonic probe inserted into a channel located on the outside surface of the balloon catheter.

FIG. 10 is a cross section view of an alternative embodiment of an ultrasonic medical device of the present invention taken along line A-A of FIG. 9.

FIG. 11 is a fragmentary side plan view of an alternative embodiment of an ultrasonic medical device of the present invention including a balloon catheter that supports a balloon and an ultrasonic probe inserted through a lumen in the balloon catheter.

FIG. 12 is a cross section view of an alternative embodiment of an ultrasonic medical device of the present invention taken along line B-B of FIG. 11.

FIG. 13 is a fragmentary side plan view of an ultrasonic medical device of the present invention located at a bend in a vasculature with an inflated balloon supported by a balloon catheter bending an ultrasonic probe along the bend in the vasculature.

FIG. 14 is a cross section view of an embodiment of an ultrasonic medical device of the present invention taken along line C-C of FIG. 13, showing a groove along an outer surface of a balloon of the ultrasonic medical device.

FIG. 15 is a cross section view of an embodiment of an ultrasonic medical device of the present invention taken along line C-C of FIG. 13, showing a smooth outer surface of a balloon of the ultrasonic medical device.

FIG. 16 is a fragmentary side plan view of an alternative embodiment of an ultrasonic probe of the present invention that includes a flexible section having a reduced diameter surrounded by sections having a larger diameter.

FIG. 17 is a fragmentary side plan view of an alternative embodiment of an ultrasonic probe of the present invention where a diameter of the ultrasonic probe increases from a flexible section to a distal end of the ultrasonic probe.

FIG. 18 is an end view of an ultrasonic medical device of the present invention with an inflated balloon supported by a balloon catheter bending an ultrasonic probe, wherein the inflated balloon covers a portion of a circumference of the balloon catheter.

FIG. 19 is an end view of an alternative embodiment of an ultrasonic medical device of the present invention with an inflated balloon supported by a balloon catheter bending an ultrasonic probe, wherein the inflated balloon surrounds the entire circumference of the balloon catheter.

FIG. 20 is a fragmentary side plan view of an ultrasonic medical device of the present invention at a bend in a vasculature adjacent to an occlusion at the bend with an inflated balloon supported by a balloon catheter bending an ultrasonic probe along the bend.

FIG. 21 is a fragmentary side plan view of an ultrasonic medical device of the present invention at a bend in a vasculature showing a plurality of transverse nodes and a plurality of transverse anti-nodes along a portion of a longitudinal axis of an ultrasonic probe.

FIG. 22 is a fragmentary side plan view of an ultrasonic medical device of the present invention at a bend in a vasculature proximal to an occlusion downstream of the bend in the vasculature.

FIG. 23 is a fragmentary side plan view of an ultrasonic medical device of the present invention at a bend in a vasculature adjacent to an occlusion upstream of the bend in the vasculature.

FIG. 24 is a fragmentary side plan view of an ultrasonic medical device of the present invention at a bend in a vasculature adjacent to multiple occlusions located proximal of the bend, at the bend and distal of the bend.

While the above-identified drawings set forth preferred embodiments of the present invention, other embodiments of the present invention are also contemplated, as noted in the discussion. This disclosure presents illustrative embodiments of the present invention by way of representation and not limitation. Numerous other modifications and embodiments can be devised by those skilled in the art which fall within the scope and spirit of the principles of the present invention.

### DETAILED DESCRIPTION

The present invention provides an apparatus and a method for bending, flexing and deflecting an ultrasonic probe while navigating through a bend of a vasculature in a body to ablate an occlusion. An ultrasonic medical device comprises the ultrasonic probe, a balloon catheter with a balloon that is supported by the balloon catheter and an at least one engaging mechanism that engages an outside surface of the balloon catheter. In a preferred embodiment of the present invention, the balloon catheter comprises two engaging mechanisms. In a preferred embodiment of the present invention, the ultrasonic probe is pre-threaded through a proximal engaging mechanism and a distal engaging mechanism. The ultrasonic medical device is moved proximal to the bend in the vasculature and the balloon is inflated, causing the ultrasonic probe to conform to the shape of the balloon while the ultrasonic probe is guided in the direction of the bend in the vasculature. As the ultrasonic probe conforms to the shape of the balloon and is guided along the bend of the vasculature, a treatment area of an occlusion destroying effect of the ultrasonic probe is expanded to ablate occlusions proximal to the bend, at the bend and distal to the bend in the vasculature.

The following terms and definitions are used herein:

“Ablate” as used herein refers to removing, clearing, destroying or taking away a biological material. “Ablation” as used herein refers to a removal, clearance, destruction, or taking away of the biological material.

“Anti-node” as used herein refers to a region of a maximum energy emitted by an ultrasonic probe at or proximal to a specific location along a longitudinal axis of the ultrasonic probe.

“Node” as used herein refers to a region of a minimum energy emitted by an ultrasonic probe at or proximal to a specific location along a longitudinal axis of the ultrasonic probe.

“Probe” as used herein refers to a device capable of propagating an energy emitted by the ultrasonic energy source along a longitudinal axis of the probe, resolving the energy into an effective cavitational energy at a specific resonance (defined by a plurality of nodes and a plurality of anti-nodes along an “active area” of the probe).

“Transverse” as used herein refers to a vibration of a probe not parallel to a longitudinal axis of the probe. A “transverse wave” as used herein is a wave propagated along the probe in which a direction of a disturbance at a plurality of points of a medium is not parallel to a wave vector.

“Biological material” as used herein refers to a collection of a matter including, but not limited to, a group of similar cells, intravascular blood clots or thrombus, fibrin, calcified plaque, calcium deposits, occlusional deposits, atherosclerotic plaque, fatty deposits, occlusions, plaque, adipose tissues, atherosclerotic cholesterol buildup, fibrous material buildup, arterial stenoses, minerals, high water content tissues, platelets, cellular debris, wastes and other occlusive materials.

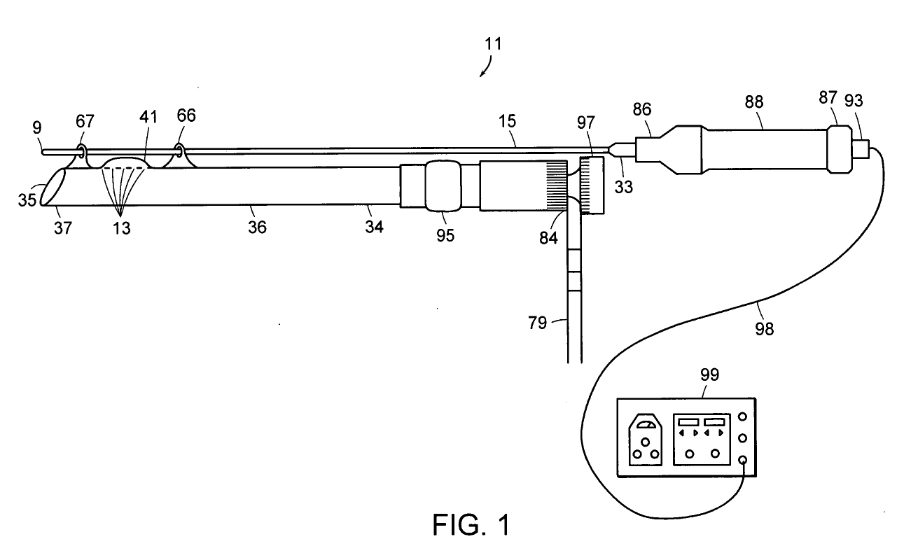

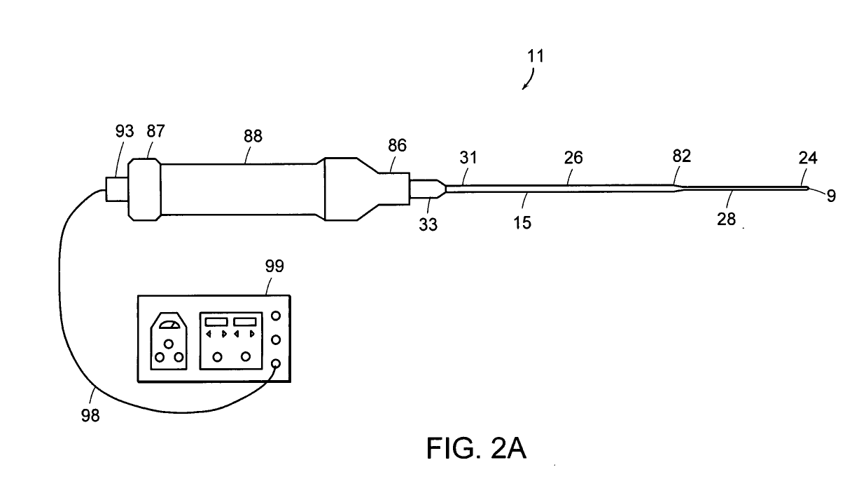

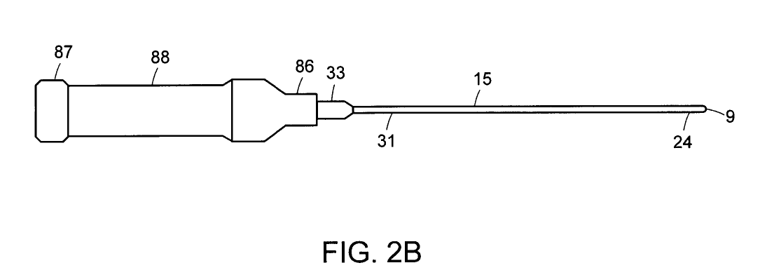

An ultrasonic medical device having an ultrasonic probe capable of bending with the aid of a balloon of the present invention is illustrated generally at 11 in FIG. 1. The ultrasonic medical device 11 includes a balloon catheter 36 that supports a balloon 41 and an ultrasonic probe 15 that is inserted through a proximal engaging mechanism 66 and a distal engaging mechanism 67. The distal engaging mechanism 67 is located at the distal end 37 of the balloon catheter 36 and the proximal engaging mechanism 66 is located proximal to the distal engaging mechanism 67. A more detailed description of the ultrasonic probe 15 is illustrated in FIG. 2A and FIG. 2B. Referring again to FIG. 1, the balloon catheter 36 has a proximal end 34, the distal end 37, a balloon catheter tip 35 and a plurality of fenestrations 13 along an outside surface of the balloon catheter 36. The balloon catheter 36 comprises an at least one engaging mechanism that engages the outside surface of the balloon catheter 36. In a preferred embodiment of the present invention, the balloon 41 is located between the proximal engaging mechanism 66 and the distal engaging mechanism 67. In a preferred embodiment of the present invention, the balloon catheter comprises the proximal engaging mechanism 66 and the distal engaging mechanism 67. In an embodiment of the present invention shown in FIG. 1, the balloon catheter 36 includes a port 84, one or more placement wings 95 and one or move valves 97. A connective tubing 79 engages the balloon catheter 36 at the port 84 and the connective tubing 79 can be opened or closed with one or more valves 97. The connective tubing 79 can be used to deliver an agent to a treatment site. An apparatus and method for an ultrasonic probe used with a pharmacological agent is disclosed in Assignee's co-pending patent application U.S. Ser. No. 10/396,914, and the entirety of the patent application is hereby incorporated herein by reference.

The balloon catheter 36 is a thin, flexible, hollow tube that is small enough to be threaded through a vein or an artery. Patients generally do not feel the movement of the balloon catheter 36 through their body. Once in place, the balloon catheter 36 allows a number of tests or other treatment procedures to be performed. Those skilled in the art will recognize that many balloon catheters known in the art can be used with the present invention and still be within the spirit and scope of the present invention.

In one embodiment of the present invention, the balloon catheter 36 comprises polytetrafluoroethylene (PTFE). In another embodiment of the present invention, the balloon catheter 36 comprises latex. In other embodiments of the present invention, the balloon catheter 36 comprises a material including, but not limited to, rubber, silicone, teflon, platinum and similar materials. Those skilled in the art will recognize that balloon catheters comprise many materials known in the art and are within the spirit and scope of the present invention.

As shown in FIG. 2A and FIG. 2B, the ultrasonic probe 15 comprises a proximal end 31 and a distal end 24 that ends in a probe tip 9. The ultrasonic probe 15 is coupled to an ultrasonic energy source or generator 99 for the production of an ultrasonic energy. A handle 88, comprising a proximal end 87 and a distal end 86, surrounds a transducer within the handle 88. The transducer, having a first end engaging the ultrasonic energy source 99 and a second end engaging the proximal end 31 of the ultrasonic probe 15, transmits the ultrasonic energy to the ultrasonic probe 15. A connector 93 and a connecting wire 98 engage the ultrasonic energy source 99 to the transducer. As shown in FIG. 2A, a diameter of the ultrasonic probe 15 decreases from a first defined interval 26 to a second defined interval 28 along the longitudinal axis of the ultrasonic probe 15 over an at least one diameter transition 82. A coupling 33 that engages the proximal end 31 of the ultrasonic probe 15 to the transducer within the handle 88 is illustrated generally in FIGS. 1, 2A and 2B. In a preferred embodiment of the present invention, the coupling 33 is a quick attachment-detachment system. An ultrasonic probe device with a quick attachment-detachment system is described in Assignee's U.S. Pat. No. 6,695,782 and co-pending patent applications U.S. Ser. No. 10/268,487 and U.S. Ser. No. 10/268,843, and the entirety of these patents and patent applications are hereby incorporated herein by reference.

FIG. 2B shows an alternative embodiment of the ultrasonic probe 15 of the present invention. In the embodiment of the present invention shown in FIG. 2B, the diameter of the ultrasonic probe 15 is approximately uniform from the proximal end 31 of the ultrasonic probe 15 to the distal end 24 of the ultrasonic probe 15.

The ultrasonic probe 15 has a stiffness that gives the ultrasonic probe 15 a flexibility so it can bend, flex and deflect. In a preferred embodiment of the present invention, the ultrasonic probe 15 is a wire. In another embodiment of the present invention, the ultrasonic probe 15 is elongated. In a preferred embodiment of the present invention, the diameter of the ultrasonic probe 15 decreases from the first defined interval 26 to the second defined interval 28. In another embodiment of the present invention, the diameter of the ultrasonic probe 15 decreases at greater than two defined intervals. In a preferred embodiment of the present invention, the transitions 82 of the ultrasonic probe 15 are tapered to gradually change the diameter from the proximal end 31 to the distal end 24 along the longitudinal axis of the ultrasonic probe 15. In another embodiment of the present invention, the transitions 82 of the ultrasonic probe 15 are stepwise to change the diameter from the proximal end 31 to the distal end 24 along the longitudinal axis of the ultrasonic probe 15. The at least one transition 82 effectively tunes the ultrasonic probe 15 to oscillate at a frequency capable of resolving the occlusion into a particulate comparable in size to red blood cells. Those skilled in the art will recognize that there can be any number of defined intervals and diameter transitions, and that the transitions can be of any shape known in the art and be within the spirit and scope of the present invention.

The probe tip 9 can be any shape including, but not limited to, bent, rounded, a ball or larger shapes. In a preferred embodiment of the present invention, the probe tip 9 is smooth to prevent damage to the vasculature. In one embodiment of the present invention, the ultrasonic energy source 99 is a physical part of the ultrasonic medical device 11. In another embodiment of the present invention, the ultrasonic energy source 99 is not a physical part of the ultrasonic medical device 11.

In a preferred embodiment of the present invention, the cross section of the ultrasonic probe 15 is approximately circular. In other embodiments of the present invention, a shape of the cross section of the ultrasonic probe 15 includes, but is not limited to, square, trapezoidal, oval, triangular, circular with a flat spot and similar cross sections. Those skilled in the art will recognize that other cross sectional geometric configurations known in the art would be within the spirit and scope of the present invention.

The ultrasonic probe 15 is inserted into the vasculature and may be disposed of after use. In a preferred embodiment of the present invention, the ultrasonic probe 15 is for a single use and on a single patient. In a preferred embodiment of the present invention, the ultrasonic probe 15 is disposable. In another embodiment of the present invention, the ultrasonic probe 15 can be used multiple times.

In a preferred embodiment of the present invention, the ultrasonic probe 15 comprises titanium or a titanium alloy. Titanium is strong, flexible, low density, and easily fabricated metal that is used as a structural material. Titanium and its alloys have excellent corrosion resistance in many environments and have good elevated temperature properties. In a preferred embodiment of the present invention, the ultrasonic probe comprises Ti-6Al-4V. The elements comprising Ti-6Al-4V and the representative elemental weight percentages of Ti-6Al-4V are titanium (about 90%), aluminum (about 6%), vanadium (about 4%), iron (maximum about 0.25%) and oxygen (maximum about 0.2%). In another embodiment of the present invention, the ultrasonic probe 15 comprises stainless steel. In another embodiment of the present invention, the ultrasonic probe 15 comprises an alloy of stainless steel. In another embodiment of the present invention, the ultrasonic probe 15 comprises aluminum. In another embodiment of the present invention, the ultrasonic probe 15 comprises an alloy of aluminum. In another embodiment of the present invention, the ultrasonic probe 15 comprises a combination of titanium and stainless steel. Those skilled in the art will recognize that the ultrasonic probe can be comprised of many materials known in the art and be within the spirit and scope of the present invention.

In a preferred embodiment of the present invention, the ultrasonic probe 15 has a small diameter. In a preferred embodiment of the present invention, the diameter of the ultrasonic probe 15 gradually decreases from the proximal end 31 to the distal end 24. In an embodiment of the present invention, the diameter of the distal end 24 of the ultrasonic probe 15 is about 0.004 inches. In another embodiment of the present invention, the diameter of the distal end 24 of the ultrasonic probe 15 is about 0.015 inches. In other embodiments of the present invention, the diameter of the distal end 24 of the ultrasonic probe 15 varies between about 0.003 inches and about 0.025 inches. Those skilled in the art will recognize an ultrasonic probe 15 can have a diameter at the distal end 24 smaller than about 0.003 inches, larger than about 0.025 inches, and between about 0.003 inches and about 0.025 inches and be within the spirit and scope of the present invention.

In an embodiment of the present invention, the diameter of the proximal end 31 of the ultrasonic probe 15 is about 0.012 inches. In another embodiment of the present invention, the diameter of the proximal end 31 of the ultrasonic probe 15 is about 0.025 inches. In other embodiments of the present invention, the diameter of the proximal end 31 of the ultrasonic probe 15 varies between about 0.003 inches and about 0.025 inches. Those skilled in the art will recognize the ultrasonic probe 15 can have a diameter at the proximal end 31 smaller than about 0.003 inches, larger than about 0.025 inches, and between about 0.003 inches and about 0.025 inches and be within the spirit and scope of the present invention.

In an embodiment of the present invention, the diameter of the ultrasonic probe 15 is approximately uniform from the proximal end 31 to the distal end 24 of the ultrasonic probe 15. In another embodiment of the present invention, the diameter of the ultrasonic probe 15 gradually decreases from the proximal end 31 to the distal end 24. In an embodiment of the present invention, the gradual change of the diameter from the proximal end 31 to the distal end 24 occurs over the at least one transition 82 with each transition 82 having an approximately equal length. In another embodiment of the present invention, the gradual change of the diameter from the proximal end 31 to the distal end 24 occurs over a plurality of transitions 82 with each transition 82 having a varying length. The transition 82 refers to a section where the diameter varies from a first diameter to a second diameter.

The physical properties (i.e., length, cross sectional shape, dimensions, etc.) and material properties (i.e., yield strength, modulus, etc.) of the ultrasonic probe 15 are selected for operation of the ultrasonic probe 15 in the transverse mode. The length of the ultrasonic probe 15 of the present invention is chosen so as to be resonant in a transverse mode. In an embodiment of the present invention, the ultrasonic probe 15 is between about 30 centimeters and about 300 centimeters in length. In an embodiment of the present invention, the ultrasonic probe 15 is a wire. Those skilled in the art will recognize an ultrasonic probe can have a length shorter than about 30 centimeters and a length longer than about 300 centimeters and be within the spirit and scope of the present invention.

The handle 88 surrounds the transducer located between the proximal end 31 of the ultrasonic probe 15 and the connector 93. In a preferred embodiment of the present invention, the transducer includes, but is not limited to, a horn, an electrode, an insulator, a backnut, a washer, a piezo microphone, and a piezo drive. The transducer is capable of an acoustic impedance transformation of electrical energy provided by the ultrasonic energy source 99 to mechanical energy. The transducer sets the operating frequency of the ultrasonic medical device 11. The transducer transmits ultrasonic energy received from the ultrasonic energy source 99 to the ultrasonic probe 15. Energy from the ultrasonic energy source 99 is transmitted along the longitudinal axis of the ultrasonic probet 15, causing the ultrasonic probe 15 to vibrate in a transverse mode. The transducer is capable of engaging the ultrasonic probe 15 at the proximal end 31 with sufficient restraint to form an acoustical mass that can propagate the ultrasonic energy provided by the ultrasonic energy source 99.

The ultrasonic energy source 99 produces a transverse ultrasonic vibration along a portion of the longitudinal axis of the ultrasonic probe 15. The ultrasonic probe 15 can support the transverse ultrasonic vibration along the portion of the longitudinal axis of the ultrasonic probe 15. The transverse mode of vibration of the ultrasonic probe 15 according to the present invention differs from an axial (or longitudinal) mode of vibration disclosed in the prior art. Rather than vibrating in an axial direction, the ultrasonic probe 15 of the present invention vibrates in a direction transverse (not parallel) to the axial direction. As a consequence of the transverse vibration of the ultrasonic probe 15, the occlusion destroying effects of the ultrasonic medical device 11 are not limited to those regions of the ultrasonic probe 15 that may come into contact with the occlusion 16. In addition, the occlusion destroying effects of the ultrasonic medical device 11 are not limited to the probe tip 9. Prior art probes undergo longitudinal vibration that is concentrated at the probe tip 9. For the present invention, as a section of the longitudinal axis of the ultrasonic probe 15 is positioned in proximity to an occlusion, a diseased area or lesion, the occlusion 16 is removed in all areas adjacent to a plurality of energetic transverse nodes and transverse anti-nodes that are produced along a portion of the longitudinal axis of the ultrasonic probe 15, typically in a region having a radius of up to about 6 mm around the ultrasonic probe 15.

The transverse ultrasonic vibration of the ultrasonic probe 15 results in a portion of the longitudinal axis of the ultrasonic probe 15 vibrated in a direction not parallel to the longitudinal axis of the ultrasonic probe 15. The transverse vibration results in movement of the longitudinal axis of the ultrasonic probe 15 in a direction approximately perpendicular to the longitudinal axis of the ultrasonic probe 15. Transversely vibrating ultrasonic probes for biological material ablation are described in the Assignee's U.S. Pat. No. 6,551,337; U.S. Pat. No. 6,652,547; U.S. Pat. No. 6,695,781 and U.S. Pat. No. 6,660,013 which further describe the design parameters for such an ultrasonic probe and its use in ultrasonic devices for an ablation, and the entirety of these patents and patent applications are hereby incorporated herein by reference.

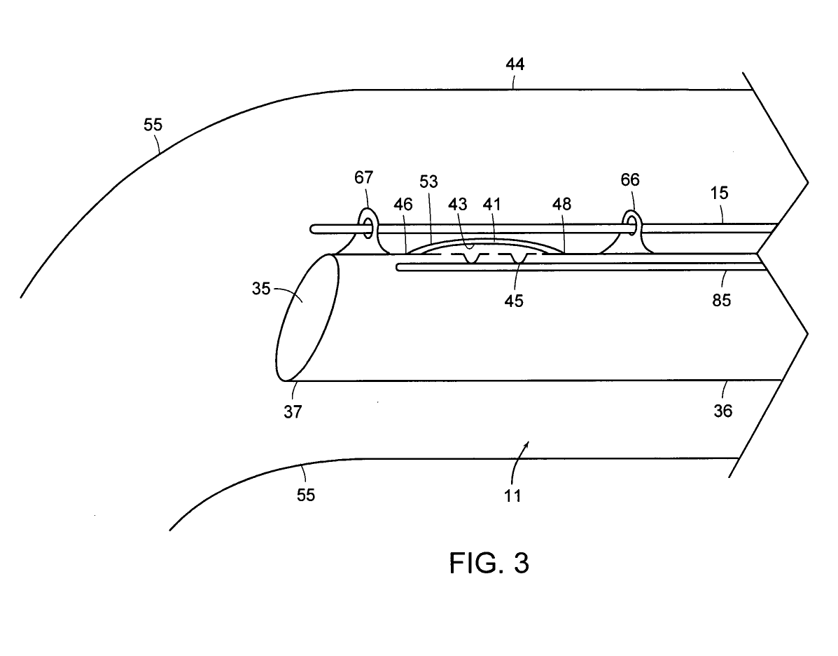

FIG. 3 shows a fragmentary view of the ultrasonic medical device 11 advanced to a bend 55 in a vasculature 44. The ultrasonic medical device 11 includes an inflation lumen 85 that is used to deliver a medium through an inflation opening 45 to engage an inner surface 43 of the balloon 41 to inflate the balloon 41. In a preferred embodiment of the present invention, an outer surface 53 of the balloon 41 does not engage the ultrasonic probe 15 when the balloon 41 is in an uninflated state. In a preferred embodiment of the present invention, the ultrasonic probe 15 is inserted into the proximal engaging mechanism 66 and the distal engaging mechanism 67 before the ultrasonic medical device 11 is inserted into the vasculature 44. In another embodiment of the present invention, the ultrasonic probe 15 is inserted into the proximal engaging mechanism 66 and the distal engaging mechanism 67 after the ultrasonic medical device 11 is inserted into the vasculature 44. FIG. 3 illustrates the balloon 41 in a deflated state and shows an intermediate step in a procedure of guiding the ultrasonic probe 15 through a bend 55 in the vasculature 44 and removing an occlusion 16 that can be either axially aligned with the vasculature 44 or not axially aligned with the vasculature 44. Several steps that precede the state shown in FIG. 3, will be discussed below.

In a preferred embodiment of the present invention, a guidewire is inserted into the vasculature 44 and moved proximal to the bend 55. In one embodiment of the present invention, the ultrasonic probe 15 is used as the guidewire. A guide catheter is placed over the proximal end of the guidewire and moved along the longitudinal axis of the guidewire. The balloon catheter 36, with the balloon 41 that is supported by the balloon catheter 36, the ultrasonic probe 15 and the inflation lumen 85 within the balloon catheter 36 are moved over the proximal end of the guidewire and moved along the longitudinal axis of the guidewire until the balloon 41 is proximal to the bend 55 in the vasculature 44. Those skilled in the art will recognize there are several ways to deliver an ultrasonic probe and a balloon catheter with a balloon supported by the balloon catheter into a vasculature that are known in the art that can be used within the spirit and scope of the present invention.

The balloon 41 engages the balloon catheter 36 at an at least one engagement position along the longitudinal axis of the balloon catheter 36. In a preferred embodiment of the present invention, the balloon 41 engages the balloon catheter 36 at a proximal engagement position 48 and a distal engagement position 46 located on the longitudinal axis of the balloon catheter 36. The balloon 41 engages the balloon catheter 36 in a manner known in the art.

In a preferred embodiment of the present invention, there are two engaging mechanisms 66, 67 located along the outside surface of the balloon catheter 36. In another embodiment of the present invention, there is a single engaging mechanism 67 located at a distal end 37 of the balloon catheter 36. In another embodiment of the present invention, there are a plurality of engaging mechanisms located along the outside surface of the balloon catheter 36. In an embodiment of the present invention, the ultrasonic probe 15 extends through the single engaging mechanism located at the distal end 37 of the balloon catheter 36. The engaging mechanism passively constrains the ultrasonic probe 15 to assist in the guiding of the ultrasonic probe 15 through the bend 55 in the vasculature 44. Those skilled in the art will recognize there can be any number of engaging mechanisms located along the outside surface of the balloon catheter 36 and be within the spirit and scope of the present invention.

The engaging mechanisms 66, 67 are smooth and contoured to prevent damage to the vasculature 44 as the balloon catheter 36 is inserted into the vasculature 44. The engaging mechanisms 66, 67 comprise openings that are contoured to prevent damage to the ultrasonic probe 15 as a portion of the longitudinal axis of the ultrasonic probe 15 engages the opening. The engaging mechanisms 66, 67 are designed to preserve the structural and ultrasonic properties of the ultrasonic probe 15 and do not effect the properties of the transverse wave that propagates down the longitudinal axis of the ultrasonic probe. In an embodiment of the present invention, the engaging mechanisms 66, 67 are located along the longitudinal axis of the balloon catheter 36 at points of a minimum energy and a minimum vibration (nodes) of the ultrasonic probe 15. The engaging mechanisms 66, 67 engage the balloon catheter 36 in manners known in the art.

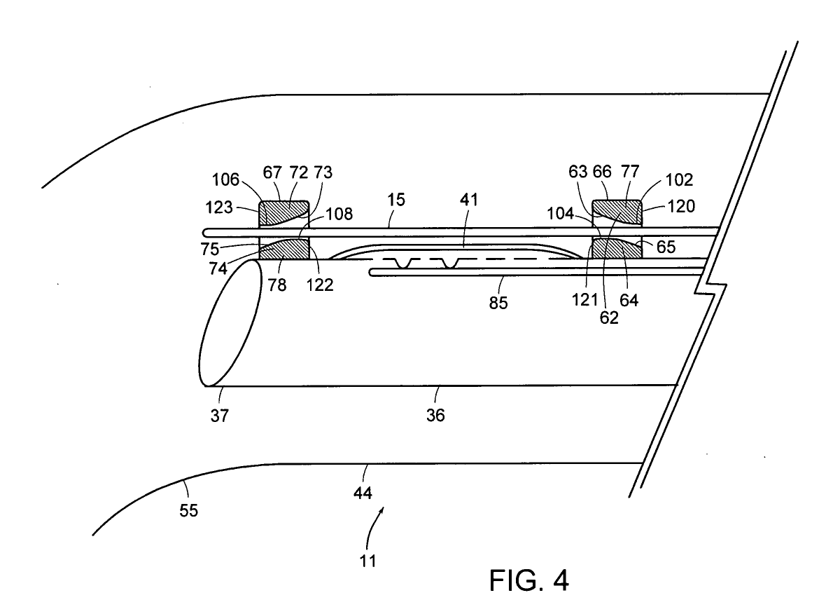

FIG. 4 illustrates a longitudinal cross section view of an embodiment of the ultrasonic medical device 11 of the present invention with the balloon 41 uninflated. FIG. 4 illustrates an embodiment of an opening of the proximal engaging mechanism 66 and an opening of the distal engaging mechanism 67. The proximal engaging mechanism 66 comprises a first face 120, a second face 121, a proximal upper section 62 and a proximal lower section 64. The proximal upper section 62 comprises a flat section 102 and a chamfered edge 63 that extends upward in the direction of the distal end 37 of the balloon catheter 36. The proximal lower section 64 comprises a flat section 104 and a chamfered edge 65 that extends upward in the direction of the distal end 37 of the balloon catheter 36. A support structure 77 surrounds the chamfered edges 63, 65 of the proximal engaging mechanism 66.

The distal engaging mechanism 67 comprises a first face 122, a second face 123, a distal upper section 72 and a distal lower section 74. The distal upper section 72 comprises a flat section 106 and a chamfered edge 73 that extends downward toward the distal end 37 of the balloon catheter 36. The distal lower section 74 comprises a flat section 108 and a chamfered edge 75 that extends downward toward the distal end 37 of the balloon catheter 36. In an embodiment of the present invention, the surface of the opening in the proximal engaging mechanism 66 and the distal engaging mechanism 67 are fully chamfered. In another embodiment of the present invention, the surface of the opening in the proximal engaging mechanism 66 and the distal engaging mechanism 67 are partially chamfered. A support structure 78 surrounds the chamfered edges 73, 75 of the distal engaging mechanism 67.

In the embodiment of the present invention shown in FIG. 4, the ultrasonic probe 15 extends between flat sections 102 and 104 of the proximal engaging mechanism 66 and between flat sections 106 and 108 of the distal engaging mechanism 67. All edges within the opening of the proximal engaging mechanism 66 and the opening of the distal engaging mechanism 67 are contoured to avoid sharp edges and corners which could cause stress concentrations and subsequently affect the mechanical and ultrasonic properties of the ultrasonic probe 15. Thus, the ultrasonic probe 15 smoothly contacts the contoured edges of the opening in the proximal engaging mechanism 66 and the distal engaging mechanism 67 without affecting the functionality of the ultrasonic probe 15. Those skilled in the art will recognize that other mechanisms to reduce stress on the ultrasonic probe are known in the art and within the spirit and scope of the present invention.

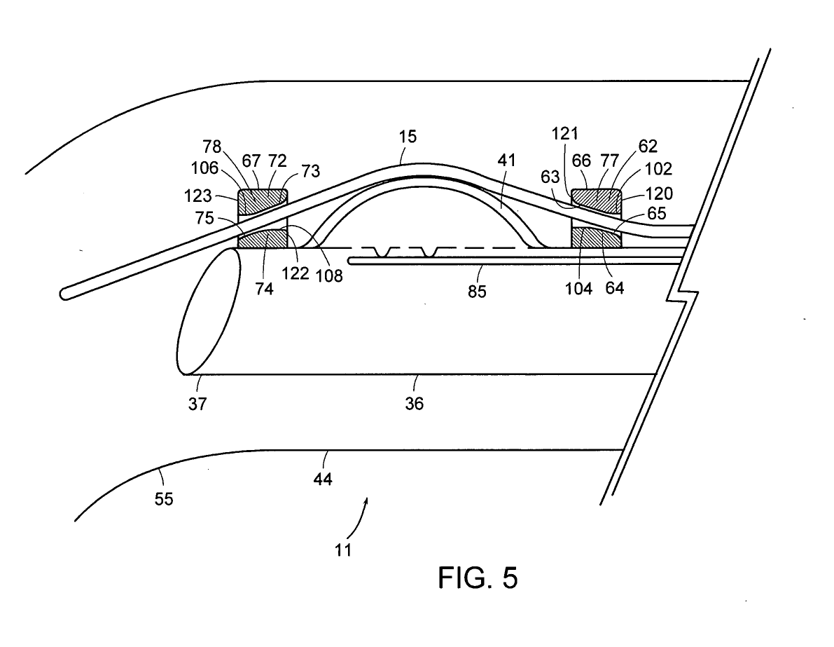

FIG. 5 illustrates a longitudinal cross section view of an embodiment of the ultrasonic medical device 11 of the present invention with the balloon 41 inflated. As the balloon 41 is inflated and engages a portion of the longitudinal axis of the ultrasonic probe 15, the ultrasonic probe 15 bends, flexes and deflects within proximal engaging mechanism 66 along the chamfered edge 65 of the proximal lower section 64 and along the chamfered edge 63 of the proximal upper section 62. In a similar manner, the ultrasonic probe 15 bends, flexes and deflects within distal engaging mechanism 67 along the chamfered edge 73 of the distal upper section 72 and along the chamfered edge 75 of the distal lower section 74. By chamfering the edges of the openings of the proximal engaging mechanism 66 and the distal engaging mechanism 67, the ultrasonic probe 15 is stabilized to control the movement of the ultrasonic probe 15 along the bend 55 in the vasculature 44. The chamfered edges 63 and 65 of the proximal engaging mechanism 66 and the chamfered edges 73 and 75 of the distal engaging mechanism 67 guide the ultrasonic probe 15 when the balloon 41 is inflated, allowing the medical professional more control to reduce the risk of injury to the vasculature 44 while moving the ultrasonic probe along the bend 55 of the vasculature 44.

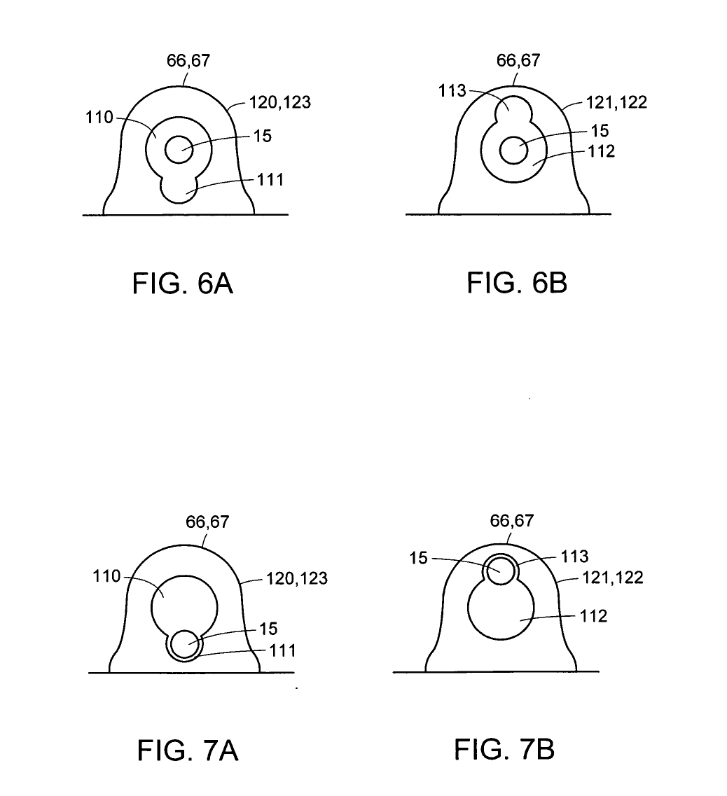

FIG. 6A shows an end view of an embodiment of a first face 120 of the proximal engaging mechanism 66 and a second face 123 of the distal engaging mechanism 67 of the present invention when the balloon 41 is uninflated. In an embodiment of the present invention shown in FIG. 6A, the first face 120 of the proximal engaging mechanism 66 and the second face 123 of the distal engaging mechanism 67 comprises a keyhole-shaped opening with an upper section 110 located on top of a smaller lower section 111. In the embodiment of the present invention shown in FIG. 6A, the ultrasonic probe 15 resides within the upper section 110 of the first face 120 of the proximal engaging mechanism 66 and the second face 123 of the distal engaging mechanism 67.

FIG. 6B shows an end view of an embodiment of a second face 121 of the proximal engaging mechanism 66 and a first face 122 of the distal engaging mechanism 67 of the present invention when the balloon 41 is uninflated. In an embodiment of the present invention shown in FIG. 6B, the second face 121 of the proximal engaging mechanism 66 and the first face 122 of the distal engaging mechanism 67 comprise a keyhole-shaped opening with a smaller upper section 113 located on top of a lower section 112. In the embodiment of the present invention shown in FIG. 6B, the ultrasonic probe 15 resides within the lower section 112 of the second face 121 of the proximal engaging mechanism 66 and the first face 122 of the distal engaging mechanism 67.

FIG. 7A shows an end view of an embodiment of the first face 120 of the proximal engaging mechanism 66 and the second face 123 of the distal engaging mechanism 67 of the present invention when the balloon 41 is inflated. FIG. 7B shows an end view of an embodiment of the second face 121 of the proximal engaging mechanism 66 and the first face 122 of the distal engaging mechanism 67. As the balloon 41 is inflated, the ultrasonic probe 15 bends, flexes and deflects as the balloon 41 engages the ultrasonic probe 15. Relative to the proximal engaging mechanism 66, the ultrasonic probe 15 moves into the smaller lower section 111 of the first face 120 of the proximal engaging mechanism 66 and the smaller upper section 113 of the second face 121 of the proximal engaging mechanism 66. Relative to the distal engaging mechanism 67, the ultrasonic probe 15 moves into the smaller upper section 113 of the first face 122 of the distal engaging mechanism 67 and the smaller lower section 111 of the second face 123 of the distal engaging mechanism 67. In effect, the ultrasonic probe 15 becomes constrained within the smaller upper section 113 and the smaller lower section 111 to allow control in moving the ultrasonic probe 15 along the bend 55 in the vasculature 44.

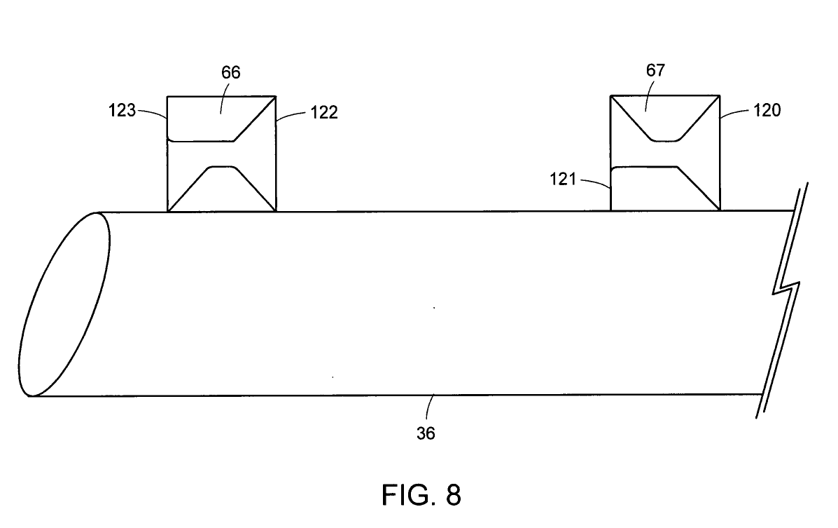

FIG. 8 shows a longitudinal cross section view of an alternative embodiment of the proximal engaging mechanism 66 and the distal engaging mechanism 67. The opening at the first face 120 of the proximal engaging mechanism 66 is larger than the opening at the second face 121 of the proximal engaging mechanism 66. The opening at the first face 120 of the proximal engaging mechanism 66 slopes to a smaller diameter along a longitudinal axis of the proximal engaging mechanism 66. The opening at the first face 122 of the distal engaging mechanism 67 is larger than the opening at the second face 123 of the distal engaging mechanism 67. The opening at the first face 122 of the distal engaging mechanism 67 slopes to a smaller diameter along a longitudinal axis of the distal engaging mechanism 67. The opening at the first face 120 of the proximal engaging mechanism 66 larger than the opening at the second face 121 of the proximal engaging mechanism guides the ultrasonic probe 15 through the proximal engaging, mechanism 66. The opening at the first face 12 of the distal engaging mechanism 67 larger than the opening at the second face 123 of the distal engaging mechanism 67 guides the ultrasonic probe 15 through the distal engaging mechanism 67.

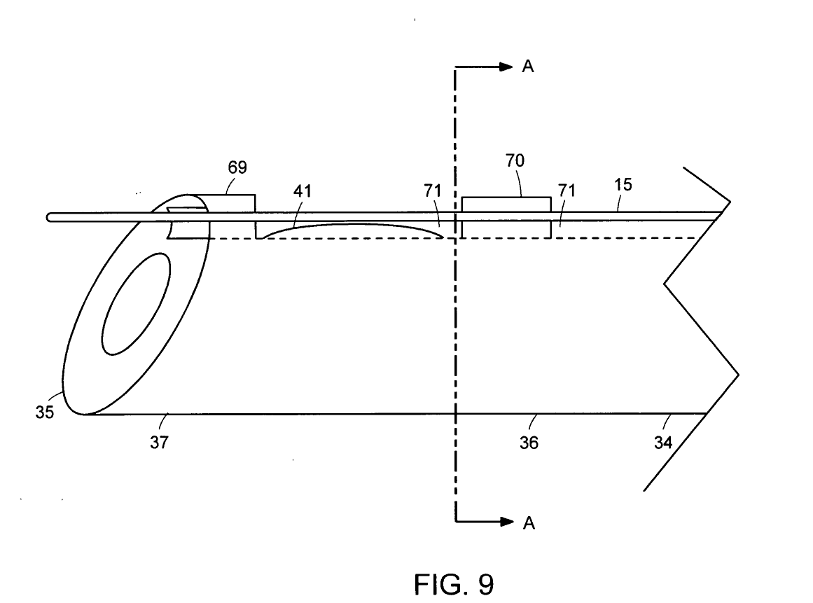

FIG. 9 shows a side view of another embodiment of the present invention, in which the ultrasonic probe 15 is inserted into a channel 71 on the outside surface along the longitudinal axis of the balloon catheter 36. The balloon 41 engages the balloon catheter 36 along a portion of the longitudinal axis of the channel 71. In a preferred embodiment of the present invention, the channel 71 comprises a proximal channel engaging support 70 and a distal channel engaging support 69. In another embodiment of the present invention, the channel 71 comprises a single channel engaging support 69 located at the distal end 37 of the balloon catheter 36. The two channel engaging supports 69, 70 are similar in function to the proximal engaging mechanism 66 and the distal engaging mechanism 67. In an embodiment of the present invention, an opening through the distal channel engaging support 69 and an opening through the proximal channel engaging support comprise chamfered edges surrounded by a support structure. The channel engaging supports 69, 70 are designed to preserve the structural and ultrasonic properties of the ultrasonic probe 15 and do not affect the properties of the transverse wave that propagates down the longitudinal axis of the ultrasonic probe. Those skilled in the art will recognize there can be many ways of passively constraining the ultrasonic probe at an at least one point along the longitudinal axis of the ultrasonic probe so the ultrasonic probe can be guided around a bend to ablate an occlusion that are within the spirit and scope of the present invention.

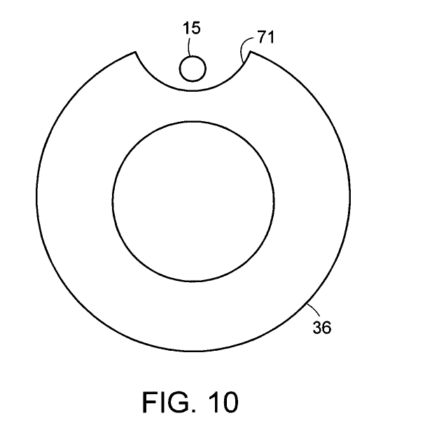

FIG. 10 shows a cross section of the embodiment of the present invention taken along line A-A in FIG. 9. The cross section shown in FIG. 10 is taken between the proximal channel engaging support 70 and the distal channel engaging support 69. The ultrasonic probe 15 is located within the channel 71.

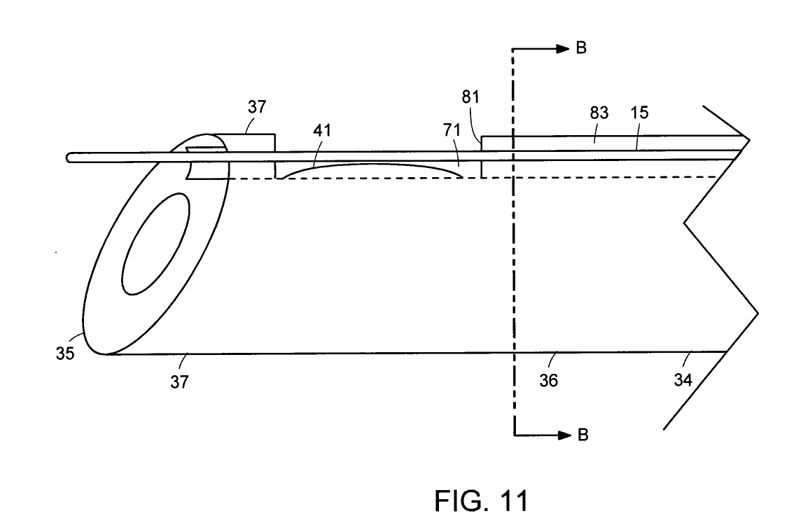

FIG. 11 shows a side view of another embodiment of the present invention, in which the ultrasonic probe 15 is inserted through a lumen 83 that extends along a longitudinal axis and through the balloon catheter 36. In the embodiment of the present invention shown in FIG. 11, the lumen 83 creates a channel 71 on the outside surface along the longitudinal axis of the balloon catheter 36. The balloon 41 and a portion of the longitudinal axis of the ultrasonic probe 15 are exposed between the distal end 37 of the balloon catheter 36 and a distal end 81 of the lumen 83.

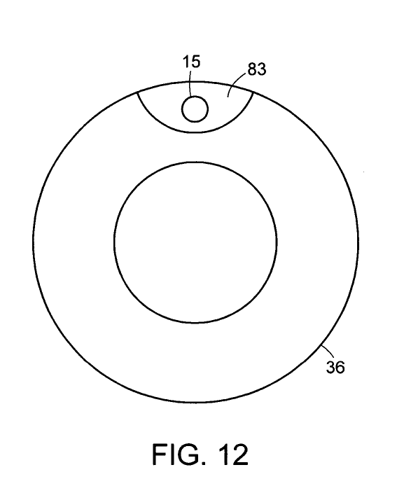

FIG. 12 shows a cross section view of the embodiment of the present invention taken along line B-B in FIG. 11. The cross section shown in FIG. 12 is taken through the lumen 83. The ultrasonic probe 15 is located within the lumen 83.

In a preferred embodiment of the present invention, a single balloon 41 is used to guide the ultrasonic probe 15 and assist in the ablation of the occlusion. In another embodiment of the present invention, two balloons 41 located along the outside surface of the balloon catheter 36 are used to guide the ultrasonic probe 15 and assist in the ablation of the occlusion. In another embodiment of the present invention, a plurality of balloons 41 are used to guide the ultrasonic probe 15 and assist in the ablation of the occlusion. Those skilled in the art will recognize there can be any number of balloons used and still be within the spirit and scope of the present invention.

In a preferred embodiment of the present invention, the balloon 41 is located between the proximal engaging mechanism 66 and the distal engaging mechanism 67. In another embodiment of the present invention, the balloon 41 extends beyond the proximal engaging mechanism 66. In another embodiment of the present invention, the balloon 41 extends beyond the distal engaging mechanism 67. In another embodiment of the present invention, the balloon 41 extends beyond the proximal engaging mechanism 66 and the distal engaging mechanism 67. Those skilled in the art will recognize the balloon can be located in several positions relative to the engaging mechanisms and be within the spirit and scope of the present invention.

In a preferred embodiment of the present invention, a single ultrasonic probe 15 is guided along the bend 55 in the vasculature 44 and used to ablate an occlusion. In another embodiment of the present invention, two ultrasonic probes 15 are guided along the bend 55 in the vasculature 44 and used to ablate the occlusion. In another embodiment of the present invention, three ultrasonic probes 15 are guided along the bend 55 in the vasculature 44 and used to ablate the occlusion. Those skilled in the art will recognize any number of ultrasonic probes can be guided along a bend in the vasculature and used to ablate an occlusion and be within the spirit and scope of the present invention.

The inflation lumen 85 is used to deliver a medium to inflate the balloon 41. In a preferred embodiment of the present invention, the medium is a liquid medium. In a preferred embodiment of the present invention, the inflation lumen 85 is located inside of the balloon catheter 36 along the longitudinal axis of the balloon catheter 36. In another embodiment of the present invention, the inflation lumen 85 is located outside of the balloon catheter 36 along the longitudinal axis of the ultrasonic probe 15. The medium moves along the insertion lumen 85 and through an at least one inflation opening 45 where the medium engages the inner surface 43 of the balloon 41, where the inner surface 43 of the balloon 41 is in communication with the inflation lumen 85. In a preferred embodiment of the present invention, the medium is a radiopaque contrast mixed with water. In another embodiment of the present invention, the medium is saline. In another embodiment of the present invention, the medium is a gas. Those skilled in the art will recognize there are many mediums used to inflate a balloon known in the art that can be used with the present invention and still be within the spirit and scope of the present invention.

An inflation mechanism is used to provide the medium into the connective tubing 79 to inflate the balloon 41 to a desired size and pressure. The medium flows along a longitudinal axis within the inflation lumen 85 and the medium moves through the at least one inflation opening 45. The balloon 41 is inflated as the medium engages the inner surface 43 of the balloon 41 and expands the balloon 41. Inflation mechanisms include, but are not limited to, syringes, screw mounted hydraulic syringes and similar devices. Those skilled in the art will recognize there are several inflation mechanisms and methods of inserting a medium into an inflation lumen known in the art that are within the spirit and scope of the present invention.

In a preferred embodiment of the present invention, the balloon 41 is a non-compliant balloon. Balloon compliance is defined as the ability of the balloon 41 to expand in diameter at various inflation pressures. In traditional balloon angioplasty procedures where a balloon is used to compress an occlusion into a wall of the vasculature, the compliance of the balloon affects the performance of the balloon when compressing an occlusion. A non-compliant balloon maintains its size and shape, even when inflated at high pressures. Non-compliant materials include, but are not limited to, polyethylene terephthalate (PET), polyurethane with nylon, duralyin and similar materials. Those skilled in the art will recognize there are many non-compliant materials known in the art that would be within the spirit and scope of the present invention.

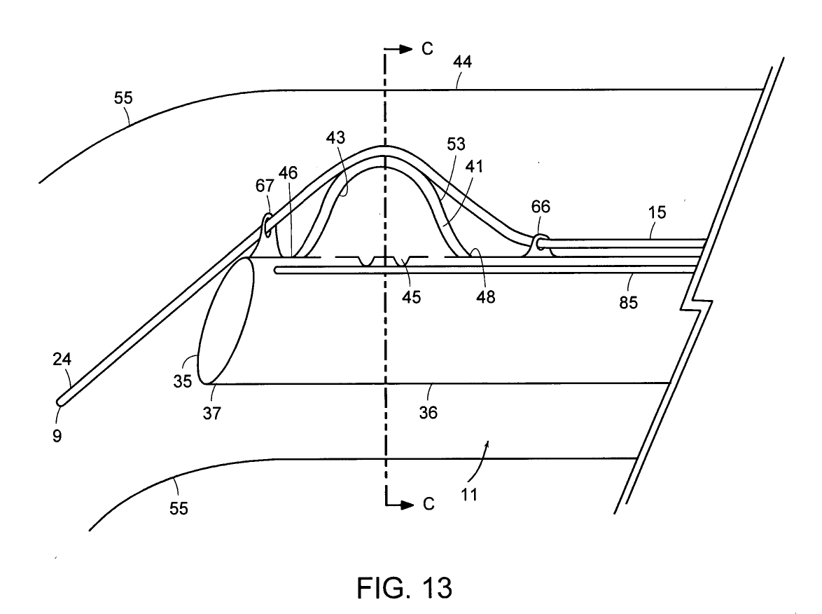

FIG. 13 shows a fragmentary side plan view of the ultrasonic medical device 11 wherein the balloon 41 is inflated and at least a portion of an outer surface 53 of the balloon 41 engages the ultrasonic probe 15. The balloon 41, upon inflation, is generally oval-shaped between the proximal engagement position 48 and the distal engagement position 46. Since the balloon 41 is oval-shaped, the balloon 41 has a large surface area which engages the ultrasonic probe 15 upon inflation. A section of the longitudinal axis of the ultrasonic probe 15 takes a non-linear shape such that the section of the longitudinal axis of the ultrasonic probe 15 between the proximal engaging mechanism 66 and the distal engaging mechanism 67 follows the contour of the outer surface 53 of the inflated balloon 41. The non-compliant inflated balloon 41 does not deform, provides support to the ultrasonic probe 15, and pushes and deflects the ultrasonic probe 15 into the non-linear shape. The distal end 24 of the ultrasonic probe 15 is guided along the bend 55 in the vasculature 44. The flexibility of the ultrasonic probe 15 allows the ultrasonic probe 15 to take the non-linear shape while maintaining the structural, material and ultrasonic properties of the ultrasonic probe 15 without any permanent deformation of the ultrasonic probe 15. The ultrasonic probe 15 comprises a material that allows the ultrasonic probe 15 to bend, deflect and flex without permanently deforming the ultrasonic probe 15. Upon deflation of the balloon 41, the ultrasonic probe 15 adopts the approximately linear shape the ultrasonic probe 15 initially had before the ultrasonic probe was bent, flexed and deflected by the inflated balloon 41. The ultrasonic probe 15 has a residual stiffness that allows the ultrasonic probe 15 to revert back to the approximately straight configuration shown in FIG. 4 when the balloon 41 is deflated. In a preferred embodiment of the present invention, the ultrasonic probe 15 does not contact the walls of the vasculature 44 as the ultrasonic probe 15 is guided along the bend 55.

In a preferred embodiment of the present invention, the tip 35 of the balloon catheter 36 is slanted so the ultrasonic probe 15 does not contact the balloon catheter 36 when the balloon 41 is inflated and the ultrasonic probe 15 is directed toward the bend 55 in the vasculature 44. In another embodiment of the present invention, the balloon 41 has a slant to prevent the ultrasonic probe 15 from contacting the balloon catheter 36 when the balloon 41 is inflated and the ultrasonic probe 15 is directed toward the bend 55 in the vasculature 44. Those skilled in the art will recognize the tip of the balloon catheter and the balloon can be shaped in many ways to prevent the ultrasonic probe from contacting the balloon catheter and be within the spirit and scope of the present invention.

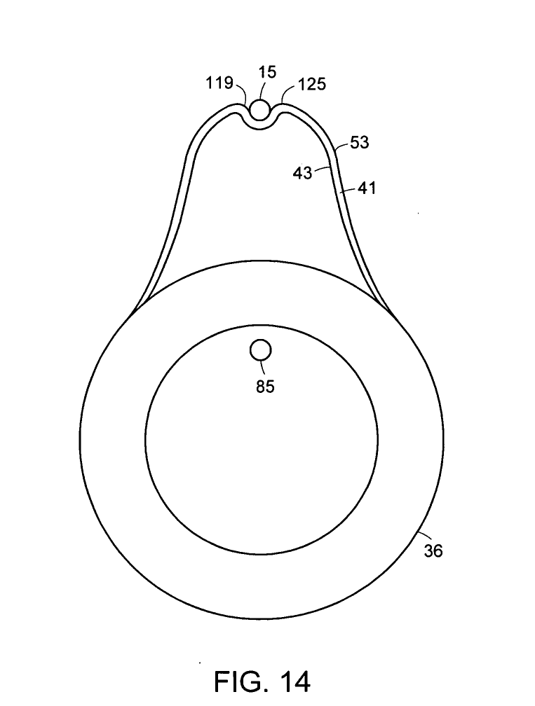

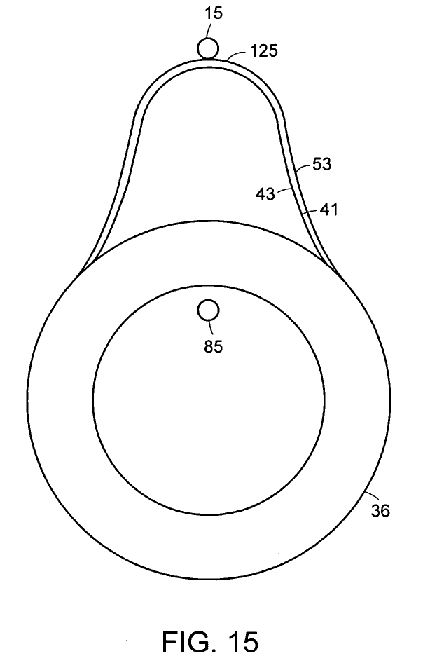

FIGS. 14 and 15 show cross sectional views of different embodiments of the ultrasonic medical device 11 of the present invention taken along line C-C of FIG. 13 when the balloon 41 is inflated. FIG. 14 illustrates an embodiment of the present invention where a top surface 125 of the balloon 41 comprises a groove 119. As shown in FIG. 14, when the balloon 41 is inflated, the ultrasonic probe 15 resides within the groove 119. The groove 119 allows for the ultrasonic probe 15 to be passively constrained to control movement of the ultrasonic probe 15 along the bend 55 in the vasculature 44. FIG. 15 shows an embodiment of the present invention where the top surface 125 of the outer surface 53 of the balloon 41 does not comprise a groove 119, but instead has the contour of the inflated balloon 41. Thus, the ultrasonic probe 15 follows the contour of the outer surface 53 of the inflated balloon 41.

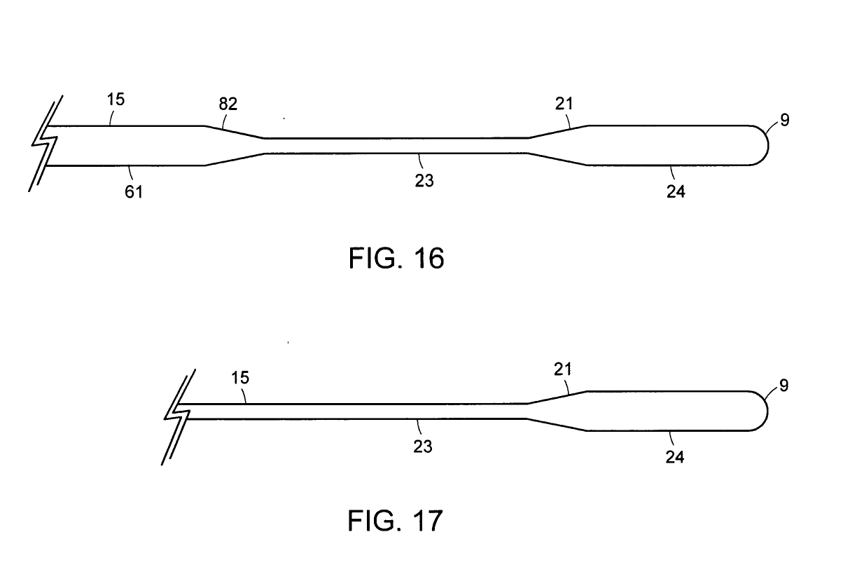

FIG. 16 shows an alternative embodiment of the present invention in which the ultrasonic probe 15 comprises a flexible section 23 having a reduced diameter that is surrounded by a proximal section 61 and the distal end 24, the proximal section 61 and the distal end 24 having a larger diameter than the flexible section 23. The diameter of the ultrasonic probe 15 decreases from the proximal section 61 to the flexible section 23 over a diameter transition 82. The diameter of the ultrasonic probe 15 increases from the flexible section 23 to the distal end 24 over a diameter transition 21. The flexible section 23 of the ultrasonic probe 15 is positioned adjacent to the balloon 41. As the balloon 41 is inflated, the balloon contacts the flexible section 23 of the ultrasonic probe 15. As the balloon 41 continues to inflate, the flexible section 23 takes on the non-linear shape of the balloon 41. The reduced diameter of the flexible section 23 improves the flexibility of the ultrasonic probe 15 and reduces the resistance of the ultrasonic probe 15 to bending. Those skilled in the art will recognize the ultrasonic probe can have any number of flexible sections and the flexible sections can be located at any location along the longitudinal axis of the ultrasonic probe and be within the spirit and scope of the present invention.

In another embodiment of the present invention, the diameter of the ultrasonic probe 15 decreases along the distal end 24 to the probe tip 9. By reducing the diameter of the ultrasonic probe 15 along the distal end 24 to the probe tip 9, the flexibility of the ultrasonic probe 15 at the distal end 24 is improved. With the balloon 41 inflated, the ultrasonic probe 15 is more easily navigated along the bend 55 in the vasculature 44 with the reduced diameter of the ultrasonic probe 15 along the distal end 24 to the probe tip 9.

FIG. 17 shows another embodiment of the present invention where the diameter of the ultrasonic probe 15 increases along the distal end 24 to the probe tip 9. The diameter of the ultrasonic probe 15 increases from the flexible section 23 over the diameter transition 21 to the distal end 24 of the ultrasonic probe 15. The ultrasonic probe 15 with the increased diameter at the distal end 24 helps decrease the amplitude of vibration at the probe tip 9.

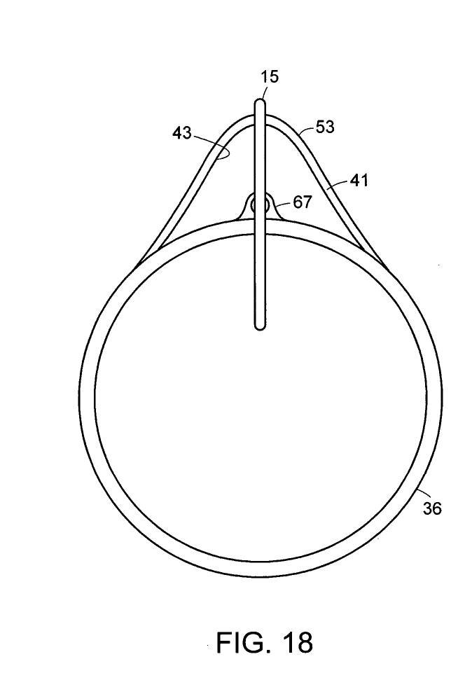

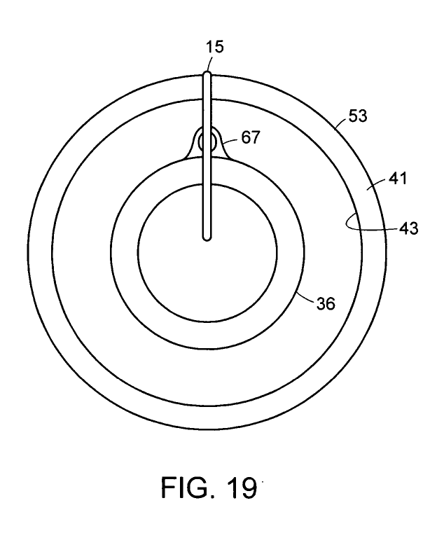

FIG. 18 shows an end view of the ultrasonic medical device 11 with the balloon 41 inflated. In a preferred embodiment of the present invention, the balloon 41 covers a portion of the circumference of the balloon catheter 36. A balloon 41 that covers a portion of the circumference of the balloon catheter 36 allows the ultrasonic probe 15 to be guided along a bend 55 in the vasculature 44 while not stressing the walls of the vasculature 44. Directional changes of the ultrasonic probe 15 in the direction of the path of the vasculature 44 are handled by rotating the balloon catheter 36 within the vasculature 44. In an alternative embodiment of the present invention, the balloon 41 covers the entire circumference of the balloon catheter 36. FIG. 19 shows an end view of an alternative embodiment of the ultrasonic medical device 11 of the present invention with the inflated balloon 41 covering the entire circumference of the balloon catheter 36. A balloon 41 that covers the entire circumference of the balloon catheter 36 helps guide the balloon catheter 36 in the vasculature 44. Those skilled in the art will recognize a balloon can cover different amounts of the circumference of the balloon catheter and be within the spirit and scope of the present invention.

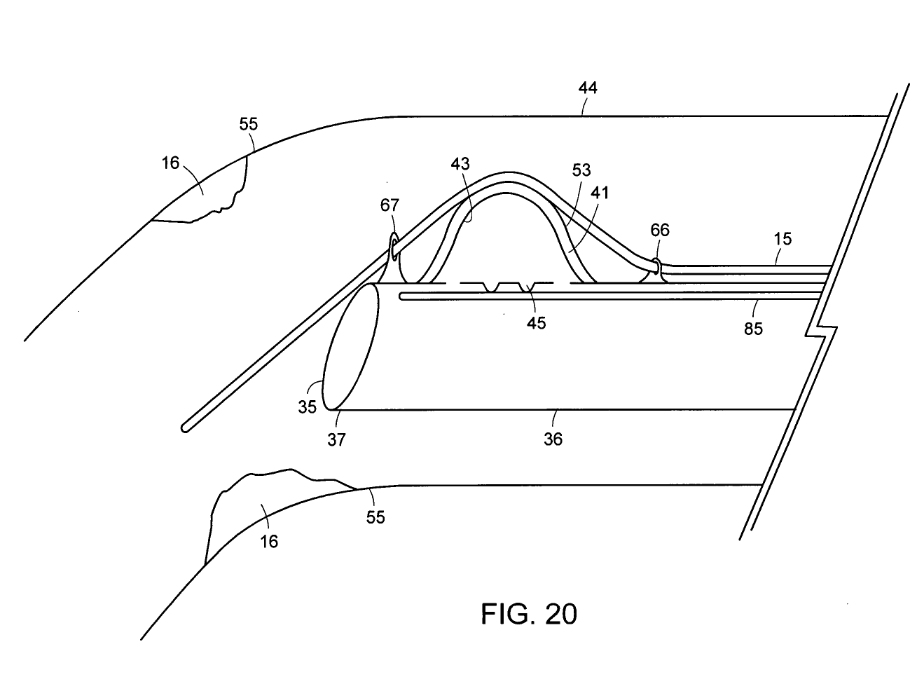

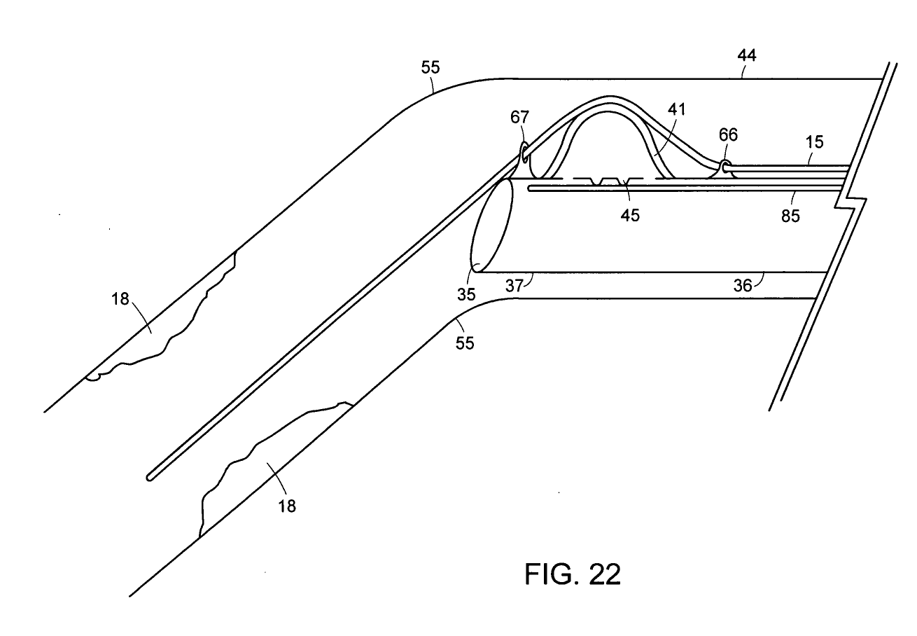

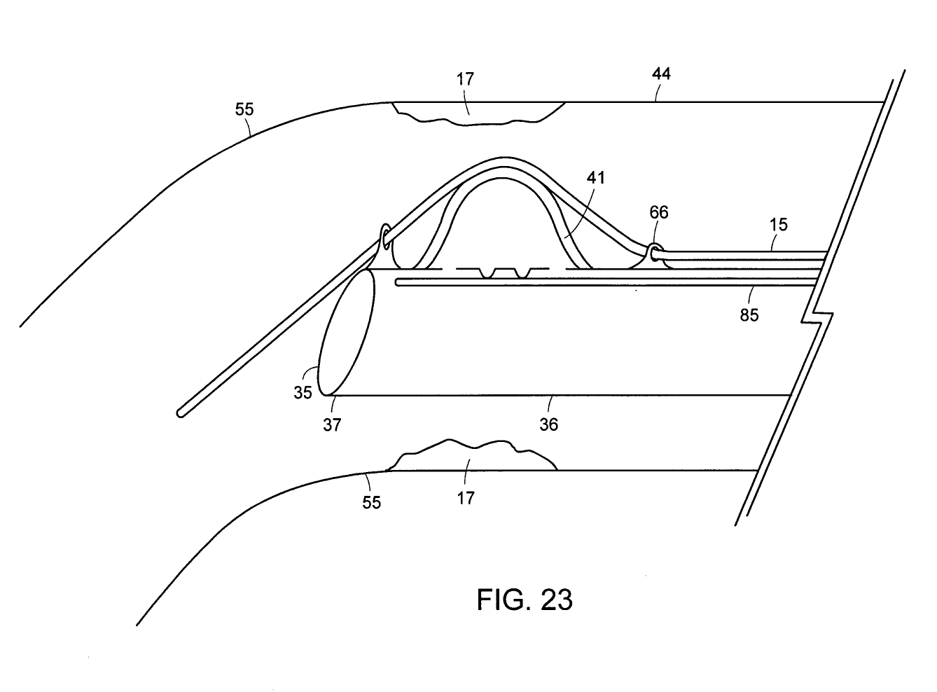

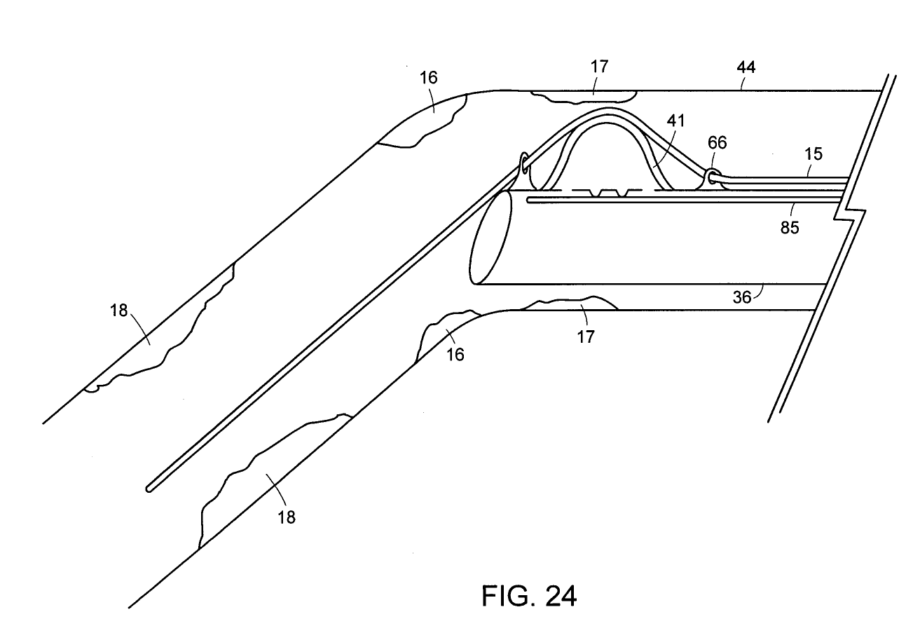

The present invention allows for the effective removal of occlusions found proximal to the bend 55 in the vasculature 44 (FIG. 23), at the bend 55 in the vasculature 44 (FIG. 20), and distal to the bend 55 in the vasculature 44 (FIG. 22). FIG. 24 illustrates that the present invention can be used to remove occlusions located at all three of these locations in the vasculature 44. The present invention increases the treatment area of an occlusion destroying effect of the ultrasonic probe 15.

FIG. 20 shows the balloon 41 inflated and the ultrasonic probe 15 guided along the bend 55 in the vasculature 44 and moved closer to an occlusion 16 that resides at the bend 55. Since the occlusion destroying effects are in a region having a radius of up to about 6 mm around the longitudinal axis of the ultrasonic probe 15, the inflation of the balloon 41 provides for effective removal of the occlusion 16 by guiding the ultrasonic probe 15 toward the occlusion 16. A probe that is inserted straight into the vasculature 44 may not be able to remove the occlusion 16 and could damage the vasculature 44. Prior art probes lack the flexibility to be moved along bends and can puncture the vasculature 44. By inserting the ultrasonic probe 15 through at least the distal engaging mechanism 67 and inflating the balloon 41, the ultrasonic probe 15 can reach occlusions at locations that are not axially aligned with the vasculature 44. The distal end 24 of the ultrasonic probe 15 moves in response to changes in the shape of the balloon 41 and the length of the balloon 41 along the longitudinal axis of the balloon catheter 36. The distal end 24 of the ultrasonic probe 15 also moves in response to how much the balloon 41 is inflated by a medium engaging an inner surface 43 of the balloon 41.

With the ultrasonic probe 15 guided along the bend 55 in the vasculature 44 toward the occlusion 16, the ultrasonic energy source 99 is activated to energize the ultrasonic probe 15. The ultrasonic energy source 99 is activated to provide a low power electric signal of between about 2 watts to about 15 watts to the transducer that is located within the handle 88. The transducer converts electrical energy provided by the ultrasonic energy source 99 to mechanical energy. The operating frequency of the ultrasonic medical device 11 is set by the transducer and the ultrasonic energy source 99 finds the resonant frequency of the transducer through a Phase Lock Loop. By an appropriately oriented and driven cylindrical array of piezoelectric crystals of the transducer, the horn creates a longitudinal wave along at least a portion of the longitudinal axis of the ultrasonic probe 15. The longitudinal wave is converted to a transverse wave along at least a portion of the longitudinal axis of the ultrasonic probe 15 through a nonlinear dynamic buckling of the ultrasonic probe 15.

As the transverse wave is transmitted along the longitudinal axis of the ultrasonic probe 15, a transverse ultrasonic vibration is created along the longitudinal axis of the ultrasonic probe 15. The ultrasonic probe 15 is vibrated in a transverse mode of vibration. The transverse mode of vibration of the ultrasonic probe 15 differs from an axial (or longitudinal) mode of vibration disclosed in the prior art. The transverse ultrasonic vibrations along the longitudinal axis of the ultrasonic probe 15 create a plurality of transverse nodes and a plurality of transverse anti-nodes along a portion of the longitudinal axis of the ultrasonic probe 15.

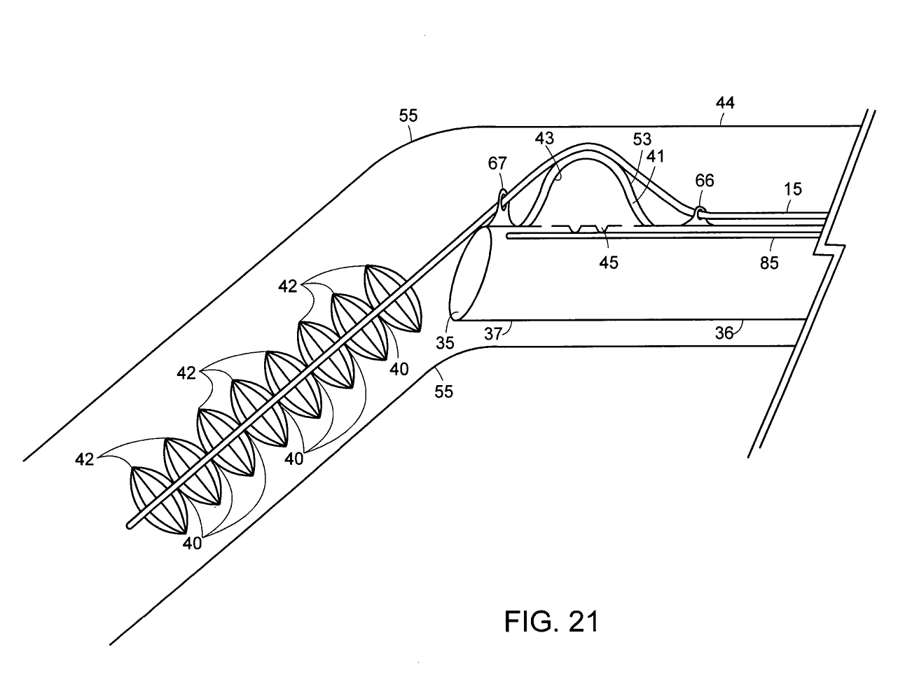

FIG. 21 shows a fragmentary side plan view of the ultrasonic medical device 11 of the present invention showing a plurality of transverse nodes 40 and a plurality of transverse anti-nodes 42 along a portion of the longitudinal axis of the ultrasonic probe 15. The transverse nodes 40 are areas of minimum energy and minimum vibration. The transverse anti-nodes 42, or areas of maximum energy and maximum vibration, also occur at repeating intervals along the portion of the longitudinal axis of the ultrasonic probe 15. The number of transverse nodes 40 and transverse anti-nodes 42, and the spacing of the transverse nodes 40 and transverse anti-nodes 42 of the ultrasonic probe 15 depend on the frequency of energy produced by the ultrasonic energy source 99. The separation of the transverse nodes 40 and transverse anti-nodes 42 is a function of the frequency, and can be affected by tuning the ultrasonic probe 15. In a properly tuned ultrasonic probe 15, the transverse anti-nodes 42 will be found at a position approximately one half of the distance between the transverse nodes 40 located adjacent to each side of the transverse anti-nodes 42. In an embodiment of the present invention where the ultrasonic probe comprises the flexible section 23, the proximal section 61 and the distal end 24, the plurality of transverse nodes 40 and the plurality of transverse anti-nodes are located along the flexible section 23, the proximal section 61 and the distal end 24 of the ultrasonic probe 15.

The transverse wave is transmitted along the longitudinal axis of the ultrasonic probe 15 and the interaction of the surface of the ultrasonic probe 15 with the medium surrounding the ultrasonic probe 15 creates an acoustic wave in the surrounding medium. As the transverse wave is transmitted along the longitudinal axis of the ultrasonic probe 15, the ultrasonic probe 15 vibrates transversely. The transverse motion of the ultrasonic probe 15 produces cavitation in the medium surrounding the ultrasonic probe 15 to ablate the occlusion 16. Cavitation is a process in which small voids are formed in a surrounding medium through the rapid motion of the ultrasonic probe 15 and the voids are subsequently forced to compress. The compression of the voids creates a wave of acoustic energy which acts to dissolve the matrix binding the occlusion 16, while having no damaging effects on healthy tissue.

The occlusion 16 is resolved into a particulate having a size on the order of red blood cells (approximately 5 microns in diameter). The size of the particulate is such that the particulate is easily discharged from the body through conventional methods or simply dissolves into the blood stream. A conventional method of discharging the particulate from the body includes transferring the particulate through the blood stream to the kidney where the particulate is excreted as bodily waste.

The transverse wave creates an acoustic pressure contour circumferentially around the ultrasonic probe 15, focusing the acoustic pressure contour to the occlusion 16. As the ultrasonic probe 15 vibrates in a transverse direction, the occlusion 16 is broken down into a particulate comparable in size to red blood cells (about 5 microns in diameter). The particulate is easily discharged from the body through conventional ways or simply dissolves into the blood stream. A conventional way of discharging the particulate from the body includes transferring the particulate through the blood stream to the kidney where the particulate is excreted as bodily waste.

The extent of the acoustic energy produced from the ultrasonic probe 15 creates a pressure wave such that the acoustic energy extends radially outward from the longitudinal axis of the ultrasonic probe 15 at the transverse anti-nodes 42 along the portion of the longitudinal axis of the ultrasonic probe 15. In this way, actual treatment time using the transverse mode ultrasonic medical device 11 according to the present invention is greatly reduced as compared to prior art methods that primarily utilize longitudinal vibration (along the axis of the probe). A distinguishing feature of the present invention is the ability to utilize ultrasonic probes of extremely small diameter compared to prior art probes.

As a consequence of the transverse ultrasonic vibration of the ultrasonic probe 15, the occlusion destroying effects of the ultrasonic medical device 11 are not limited to those regions of the ultrasonic probe 15 that may come into contact with the occlusion 16. Rather, as a section of the longitudinal axis of the ultrasonic probe 15 is positioned in proximity to the occlusion 16, the occlusion 16 is removed in all areas adjacent to the plurality of energetic transverse nodes 40 and transverse anti-nodes 42 that are produced along the portion of the length of the longitudinal axis of the ultrasonic probe 15, typically in a region having a radius of up to about 6 mm around the ultrasonic probe 15.

A novel feature of the present invention is the ability to utilize ultrasonic probes 15 of extremely small diameter compared to prior art probes, without loss of efficiency, because the occlusion fragmentation process is not dependent on the area of the probe tip 9. Highly flexible ultrasonic probes 15 can therefore be designed to mimic device shapes that enable facile insertion into occlusion areas or extremely narrow interstices that contain the occlusion 16. Another advantage provided by the present invention is the ability to rapidly move the occlusion 16 from large areas within cylindrical or tubular surfaces. The number of transverse nodes 40 and transverse anti-nodes 42 occurring along the longitudinal axis of the ultrasonic probe 15 is modulated by changing the frequency of energy supplied by the ultrasonic energy source 99. The exact frequency, however, is not critical and the ultrasonic energy source 99 run at, for example, about 20 kHz is sufficient to create an effective number of occlusion 16 destroying transverse anti-nodes 42 along the longitudinal axis of the ultrasonic probe 15. The low frequency requirement of the present invention is a further advantage in that the low frequency requirement leads to less damage to healthy tissue. Those skilled in the art understand it is possible to adjust the dimensions of the ultrasonic probe 15, including diameter, length and distance to the ultrasonic energy source 99, in order to affect the number and spacing of the transverse nodes 40 and transverse anti-nodes 42 along a portion of the longitudinal axis of the ultrasonic probe 15.

The present invention allows the use of ultrasonic energy to be applied to the occlusion 16 selectively, because the ultrasonic probe 15 conducts energy across a frequency range from about 10 kHz through about 100 kHz. The amount of ultrasonic energy to be applied to a particular treatment site is a function of the amplitude and frequency of vibration of the ultrasonic probe 15. In general, the amplitude or throw rate of the energy is in the range of about 25 microns to about 250 microns, and the frequency in the range of about 10 kHz to about 100 kHz. In a preferred embodiment of the present invention, the frequency of ultrasonic energy is from about 20 kHz to about 35 kHz. Frequencies in this range are specifically destructive of occlusions 16 including, but not limited to, hydrated (water-laden) tissues such as endothelial tissues, while substantially ineffective toward high-collagen connective tissue, or other fibrous tissues including, but not limited to, vascular tissues, epidermal, or muscle tissues.

The inflation of the balloon 41 bends the ultrasonic probe 15 to increase a surface area of the ultrasonic probe 15 in communication with the occlusion 16. The ultrasonic probe 15 is guided in a direction where a greater surface area of the ultrasonic probe 15 is in communication with the occlusion 16 when compared to a probe that is introduced straight into the vasculature 44. The ultrasonic probe 15 is able to transfer ultrasonic energy in a bent configuration in addition to a straight configuration. The ultrasonic probe 15 vibrates in a plurality of bent configurations and can simultaneously ablate occlusions before, at and after the bend in the bent configuration. The longitudinal axis of the ultrasonic probe 15 is positioned closer to the occlusion 16 by the inflation of the balloon 41 to bend the ultrasonic probe 15. The inflation of the balloon 41 provides a large active area of the ultrasonic probe 15 for ablation of the occlusion 16 and maximizes a radial span of the ultrasonic probe 15 within the vasculature 44. As the ultrasonic probe 15 conforms to the shape of the inflated balloon 41 and is directed along the bend 55 in the vasculature 44, the treatment area of the ultrasonic probe 15 is expanded, allowing for the occlusion destroying effects of the ultrasonic probe 15 to be focused on the occlusion 16.

In order to effectively remove the occlusion 16, the ultrasonic probe 15 can be moved within the vasculature 44. In one embodiment of the present invention, the ultrasonic probe 15 is moved back and forth along the occlusion 16. In another embodiment of the present invention, the ultrasonic probe 15 is swept along the occlusion 16. In another embodiment of the present invention, the ultrasonic probe 15 is rotated along the occlusion 16. In another embodiment of the present invention, the ultrasonic probe 15 is twisted along the occlusion 16. Those skilled in the art will recognize an ultrasonic probe can be moved in many ways and still be within the spirit and scope of the present invention.

The present invention provides for occlusion ablation at locations in addition to the occlusion 16 at the bend 55 in the vasculature 44. As the ultrasonic probe 15 is guided along the bend 55 in the vasculature 44, the ultrasonic probe 15 can treat occlusions downstream of the occlusion 16 at the bend 55 in the vasculature 44. The ultrasonic probe 15 can treat occlusions before the bend 55 in the vasculature 44.

FIG. 22 illustrates the ultrasonic probe 15 moved further along the bend 55 in the vasculature 44 and proximal to an occlusion 18 located along the portion of the vasculature 44 further downstream of the bend 55. In a preferred embodiment of the present invention, the occlusion comprises a biological material. In the same ablation methods as previously discussed, the occlusion 18 is resolved into a particulate comparable in size to red blood cells and is discharged from the body through conventional ways or simply dissolves into the blood stream. Prior art probes that are straight would not be capable of navigating the bend to be moved proximal to the occlusion. Prior art probes lack the flexibility to follow the bend in the vasculature and could puncture the vasculature. Prior art probes that are shaped are unable to be navigated through a bend in the vasculature and moved proximal to the occlusion. The present invention solves these problems of prior art probes and allows ablation of an occlusion located downstream of the bend.

FIG. 23 shows the ultrasonic probe 15 in communication with an occlusion 17 located before the bend 55 in the vasculature 44. In FIG. 23, the occlusion 17 is located between the proximal engaging mechanism 66 and the distal engaging mechanism 67. In a preferred embodiment of the present invention, the occlusion 17 comprises a biological material. As the balloon 41 is inflated, the outer surface 53 of the balloon 41 engages the ultrasonic probe 15 and moves a segment of the longitudinal axis of the ultrasonic probe 15 between the proximal engaging mechanism 66 and the distal engaging mechanism 67 closer to the occlusion 17. As discussed above, the ultrasonic probe 15 resolves the occlusion 18 into a particulate comparable in size to red blood cells which is discharged from the body through conventional ways or dissolves into the blood stream. FIG. 24 shows the ultrasonic probe 15 in communication with a plurality of occlusions located before, at and downstream of the bend 55 in the vasculature 44. The present invention can be used to ablate the occlusion 17 before the bend 55, the occlusion 16 at the bend 55 and the occlusion 18 further downstream of the bend 55 in the vasculature 44. By bending the ultrasonic probe 15 with the aid of the balloon 41, the ultrasonic probe 15 can ablate the occlusion 16 in a plurality of bent configurations. The inflation of the balloon 41 provides for an increased treatment area of the occlusion destroying effects of the ultrasonic probe 15. The plurality of occlusions 16, 17, 18 are resolved into a particulate comparable in size to red blood cells in a time efficient manner.

The present invention provides a method of moving an ultrasonic probe 15 in a vasculature 44 to ablate an occlusion in a vasculature 44. The ultrasonic probe 15 is inserted through a proximal engaging mechanism 66 located on the outside surface 53 of the balloon catheter 36. The ultrasonic probe 15 is moved over the outer surface 53 of the balloon 41 and through the distal engaging mechanism 67 located on the outside surface of the balloon catheter 36. The balloon catheter 36 is advanced until the balloon 41 is adjacent to the bend 55 in the vasculature 44. The balloon 41 is inflated, causing the outer surface 53 of the balloon 41 to engage the ultrasonic probe 15, thereby causing the ultrasonic probe 15 to bend between the proximal engaging mechanism 66 and the distal engaging mechanism 67. The ultrasonic probe 15 is advanced along the outer surface 53 of the balloon 41 to move the ultrasonic probe 15 along the bend 55 in the vasculature 44 and proximal to the occlusion. The ultrasonic probe 15 is energized to produce a transverse ultrasonic vibration to ablate the occlusion 16 at the bend 55 in the vasculature 44 in the bent configuration of the ultrasonic probe 15.

The present invention also provides a method of moving an ultrasonic probe 15 capable of adopting a non-linear shape along the bend 55 within the vasculature 44 of the body without damaging the vasculature 44 to remove the occlusion. The present invention provides a balloon catheter 36 having a balloon 41 in communication with an outside surface 53 of the balloon catheter 36 and the ultrasonic probe 15 extending along the outer surface 53 of the balloon 41. The balloon 41 is inflated and a surface area of the ultrasonic probe 15 in communication with the occlusion is increased. The ultrasonic probe 15 is moved along the outer surface 53 of the balloon 41 and along the bend 55 in the vasculature 44 and further downstream of the bend 55. The ultrasonic energy source 99 is activated to provide an ultrasonic energy to the ultrasonic probe 15 to remove the occlusions along the vasculature 44.

The present invention provides a method of increasing a treatment area of an occlusion destroying effect of the ultrasonic probe 15. By inflating the balloon 41 and guiding the ultrasonic probe 15 along the bend 55 in the vasculature 44, a radial span of the ultrasonic probe 15 is increased and the ultrasonic probe 15 is moved closer to the occlusions before the bend 55, at the bend 55 and downstream of the bend 55 in the vasculature 44. The present invention focuses the occlusion destroying effects of the ultrasonic probe 15 on the occlusions.

In an alternative embodiment of the present invention, the ultrasonic probe 15 is vibrated in a torsional mode. In the torsional mode of vibration, a portion of the longitudinal axis of the ultrasonic probe 15 comprises a radially asymmetric cross section and the length of the ultrasonic probe 15 is chosen to be resonant in the torsional mode. In the torsional mode of vibration, a transducer transmits ultrasonic energy received from the ultrasonic energy source 99 to the ultrasonic probe 15, causing the ultrasonic probe 15 to vibrate torsionally. The ultrasonic energy source 99 produces the electrical energy that is used to produce a torsional vibration along the longitudinal axis of the ultrasonic probe 15. The torsional vibration is a torsional oscillation whereby equally spaced points along the longitudinal axis of the ultrasonic probe 15 including the probe tip 9 vibrate back and forth in a short arc about the longitudinal axis of the ultrasonic probe 15. A section proximal to each of a plurality of torsional nodes and a section distal to each of the plurality of torsional nodes are vibrated out of phase, with the proximal section vibrated in a clockwise direction and the distal section vibrated in a counterclockwise direction, or vice versa. The torsional vibration results in an ultrasonic energy transfer to the biological material with minimal loss of ultrasonic energy that could limit the effectiveness of the ultrasonic medical device 11. The torsional vibration produces a rotation and a counterrotation along the longitudinal axis of the ultrasonic probe 15 that creates the plurality of torsional nodes and a plurality of torsional anti-nodes along a portion of the longitudinal axis of the ultrasonic probe 15 resulting in cavitation along the portion of the longitudinal axis of the ultrasonic probe 15 comprising the radially asymmetric cross section in a medium surrounding the ultrasonic probe 15 that ablates the biological material. An apparatus and method for an ultrasonic medical device operating in a torsional mode is described in Assignee's co-pending patent application U.S. Ser. No. 10/774,985, and the entirety of this application is hereby incorporated herein by reference.

In another embodiment of the present invention, the ultrasonic probe 15 is vibrated in a torsional mode and a transverse mode. A transducer transmits ultrasonic energy from the ultrasonic energy source 99 to the ultrasonic probe 15, creating a torsional vibration of the ultrasonic probe 15. The torsional vibration induces a transverse vibration along an active area of the ultrasonic probe 15, creating a plurality of nodes and a plurality of anti-nodes along the active area that result in cavitation in a medium surrounding the ultrasonic probe 15. The active area of the ultrasonic probe 15 undergoes both the torsional vibration and the transverse vibration.

Depending upon physical properties (i.e., length, diameter, etc.) and material properties (i.e., yield strength, modulus, etc.) of the ultrasonic probe 15, the transverse vibration is excited by the torsional vibration. Coupling of the torsional mode of vibration and the transverse mode of vibration is possible because of common shear components for the elastic forces. The transverse vibration is induced when the frequency of the transducer is close to a transverse resonant frequency of the ultrasonic probe 15. The combination of the torsional mode of vibration and the transverse mode of vibration is possible because for each torsional mode of vibration, there are many close transverse modes of vibration. By applying tension on the ultrasonic probe 15, for example by bending the ultrasonic probe 15, the transverse vibration is tuned into coincidence with the torsional vibration. The bending causes a shift in frequency due to changes in tension. In the torsional mode of vibration and the transverse mode of vibration, the active area of the ultrasonic probe 15 is vibrated in a direction not parallel to the longitudinal axis of the ultrasonic probe 15 while equally spaced points along the longitudinal axis of the ultrasonic probe 15 in a proximal section vibrate back and forth in a short arc about the longitudinal axis of the ultrasonic probe 15. An apparatus and method for an ultrasonic medical device operating in a transverse mode and a torsional mode is described in Assignee's co-pending patent application U.S. Ser. No. 10/774,898, and the entirety of this application is hereby incorporated herein by reference.

The present invention provides an apparatus and a method of bending, flexing and deflecting an ultrasonic probe 15 along the vasculature 44 to increase a surface area of the ultrasonic probe 15 in communication with a plurality of occlusions along the vasculature 44. The present invention provides an apparatus and a method of guiding the ultrasonic probe 15 along the bend 55 of the vasculature 44 to remove occlusions that is simple, user friendly, reliable, time efficient, cost effective and does not harm the vasculature.

All patents, patent applications, and published references cited herein are hereby incorporated herein by reference in their entirety. While this invention has been particularly shown and described with references to preferred embodiments thereof, it will be understood by those skilled in the art that various changes in form and details may be made therein without departing from the scope of the invention encompassed by the appended claims.

1. An ultrasonic medical device comprising:
a balloon catheter having a proximal end, a distal end and a longitudinal axis therebetween;
an inflation lumen located along the longitudinal axis of the balloon catheter;
a balloon supported by the balloon catheter, an inner surface of the balloon in communication with the inflation lumen; and
an ultrasonic probe located along an outside surface of the balloon catheter, the ultrasonic probe engaging an outer surface of the balloon.

2. The device of claim 1 wherein the inflation lumen is located on the outside surface of the balloon catheter.

3. The device of claim 1 wherein the inflation lumen is located inside of the balloon catheter.

4. The device of claim 1 wherein the balloon catheter further comprises a proximal engaging mechanism located along the outside surface of the balloon catheter.

5. The device of claim 4 wherein the balloon catheter further comprises a distal engaging mechanism located along the outside surface of the balloon catheter.

6. The device of claim 1 wherein the balloon catheter further comprises a plurality of engaging mechanisms located along the outside surface of the balloon catheter.

7. The device of claim 1 wherein the ultrasonic probe extends through an at least one engaging mechanism located along the outside surface of the balloon catheter.

8. The device of claim 1 further comprising an at least one engaging mechanism located at the distal end of the balloon catheter.

9. The device of claim 1 wherein a portion of the balloon is located between a proximal engaging mechanism located along the outside surface of the balloon catheter and a distal engaging mechanism located along the outside surface of the balloon catheter.

10. The device of claim 1 further comprising a channel located along the outside surface of the longitudinal axis of the balloon catheter.

11. The device of claim 10 wherein the ultrasonic probe resides in the channel.

12. The device of claim 1 wherein the balloon is non-compliant.

13. The device of claim 1 further comprising a second ultrasonic probe located along the outside surface of the balloon catheter.

14. An ultrasonic medical device comprising:
a balloon catheter comprising an at least one engaging mechanism located along an outside surface of the balloon catheter;
a balloon having an outer surface and an inner surface, the balloon engaging the outside surface of the balloon catheter;
an elongated ultrasonic probe extending through the at least one engaging mechanism and engaging the outer surface of the balloon; and
an inflation lumen in communication with the balloon, the inflation lumen located along the longitudinal axis of the balloon catheter.

15. The device of claim 14 wherein the inflation lumen is located inside of the balloon catheter.

16. The device of claim 14 wherein the inflation lumen is located on the outside surface of the balloon catheter.

17. The device of claim 14 wherein one of the at least one engaging mechanisms is located at a distal end of the balloon catheter.

18. The device of claim 14 wherein the elongated ultrasonic probe engages the outer surface of the balloon when the balloon is inflated.

19. The device of claim 14 wherein the balloon is non-compliant.

20. The device of claim 14 wherein an injection of a medium through the inflation lumen expands the balloon to guide the elongated ultrasonic probe.

21. The device of claim 14 wherein a distal end of the elongated ultrasonic probe moves in response to changes in a shape of the balloon.

22. The device of claim 14 wherein the balloon is located over a portion of a circumference of the balloon catheter.

23. The device of claim 14 wherein a portion of the balloon is located adjacent to the at least one engaging mechanism.

24. A method of moving an ultrasonic probe along a bend in a vasculature to ablate an occlusion in the vasculature comprising:
inserting the ultrasonic probe through a proximal engaging mechanism located on an outside surface of a balloon catheter;
moving the ultrasonic probe over an outer surface of a balloon supported by the balloon catheter and through a distal engaging mechanism located on the outside surface of the balloon catheter;
advancing the balloon catheter until the balloon is adjacent to the bend in the vasculature;
inflating the balloon causing the outer surface of the balloon to engage the ultrasonic probe, thereby causing the ultrasonic probe to bend between the proximal engaging mechanism and the distal engaging mechanism;
advancing the ultrasonic probe along the outer surface of the balloon to move the ultrasonic probe along the bend in the vasculature adjacent to the occlusion; and
energizing the ultrasonic probe to ablate the occlusion at the bend in the vasculature.

25. The method of claim 24 further comprising inflating the balloon through an injection of a medium in an inflation lumen located along a longitudinal axis of the balloon catheter.

26. The method of claim 25 further comprising engaging the medium to an inner surface of the balloon through an at least one inflation opening along a longitudinal axis of the inflation lumen.

27. The method of claim 24 further comprising changing a shape of the balloon to move a distal end of the ultrasonic probe.

28. The method of claim 24 further comprising engaging a medium to an inner surface of the balloon to move a distal end of the ultrasonic probe.

29. The method of claim 24 further comprising modifying a length of the balloon along a longitudinal axis of the balloon catheter to move a distal end of the ultrasonic probe.

30. The method of claim 24 further comprising increasing a surface area of the ultrasonic probe in communication with the occlusion through an inflation of the balloon.

31. The method of claim 24 further comprising inflating the balloon to provide a large active area for ablation of the occlusion.

32. The method of claim 24 further comprising inflating the balloon to maximize a radial span of the ultrasonic probe within the vasculature.

33. The method of claim 24 further comprising inflating the balloon to expand a treatment area of the ultrasonic probe.

34. The method of claim 24 further comprising inflating the balloon to focus an occlusion destroying effect of the ultrasonic probe.

35. The method of claim 24 further comprising inflating the balloon to support the ultrasonic probe.

36. The method of claim 24 further comprising moving the ultrasonic probe back and forth along the occlusion.

37. The method of claim 24 further comprising sweeping the ultrasonic probe along the occlusion.

38. The method of claim 24 further comprising rotating the ultrasonic probe along the occlusion.

39. The method of claim 24 further comprising twisting the ultrasonic probe along the occlusion.

40. The method of claim 24 wherein the balloon is a non-compliant balloon.

41. A method of moving a flexible ultrasonic probe capable of having a non-linear shape along a bend within a vasculature of a body to remove a biological material comprising:
providing a balloon catheter having a balloon in communication with an outside surface of the balloon catheter and the flexible ultrasonic probe extending along an outer surface of the balloon;
inflating the balloon to increase a surface area of the flexible ultrasonic probe in communication with the biological material;
moving the flexible ultrasonic probe along the outer surface of the balloon to move the flexible ultrasonic probe along the bend in the vasculature toward the biological material; and
activating an ultrasonic energy source to provide an ultrasonic energy to the ultrasonic probe to remove the biological material.

42. The method of claim 41 further comprising inserting the flexible ultrasonic probe through an engaging mechanism located on the outside surface at a distal end of the balloon catheter.

43. The method of claim 41 further comprising inserting the flexible ultrasonic probe through a plurality of engaging mechanisms located on the outside surface of the balloon catheter.

44. The method of claim 41 further comprising injecting a medium in an inflation lumen to inflate the balloon.

45. The method of claim 44 further comprising engaging the medium to the inner surface of the balloon through an inflation opening along a longitudinal axis of the inflation lumen.

46. The method of claim 41 further comprising bending the flexible ultrasonic probe at an angle to a longitudinal axis of the balloon catheter.

47. The method of claim 41 further comprising changing a shape of the balloon to move a distal end of the flexible ultrasonic probe.

48. The method of claim 41 further comprising engaging a medium to an inner surface of the balloon to move a distal end of the flexible ultrasonic probe.

49. The method of claim 41 further comprising modifying a length of the balloon along a longitudinal axis of the balloon catheter to move a distal end of the flexible ultrasonic probe.

50. The method of claim 41 further comprising locating the balloon over a portion of a circumference of the balloon catheter.

51. The method of claim 41 further comprising inflating the balloon to increase a radial span of the flexible ultrasonic probe within the vasculature.

52. The method of claim 41 further comprising inflating the balloon to expand a treatment area of a biological material destroying effect of the flexible ultrasonic probe.

53. A balloon catheter comprising:
a proximal end, a distal end and a longitudinal axis therebetween;
an inflation lumen located along the longitudinal axis of the balloon catheter;
a balloon supported by the balloon catheter, an inner surface of the balloon in communication with the inflation lumen; and
a distal engaging mechanism extending from an outside surface of the distal end of the balloon catheter.

54. The balloon catheter of claim 53 wherein the distal engaging mechanism comprises an opening having chamfered edges surrounded by a support structure.

55. The balloon catheter of claim 54 wherein the chamfered edges of the opening of the distal engaging mechanism extend downward in the direction of the distal end of the balloon catheter.

56. The balloon catheter of claim 53 further comprising a proximal engaging mechanism extending from the outside surface of the balloon catheter proximal to the distal engaging mechanism.

57. The balloon catheter of claim 56 wherein the proximal engaging mechanism comprises an opening having chamfered edges surrounded by a support structure.

58. The balloon catheter of claim 57 wherein the chamfered edges of the opening of the proximal engaging mechanism extend upward in the direction of the distal end of the balloon catheter.

59. The balloon catheter of claim 53 wherein an opening in the distal engaging mechanism has a keyhole shape with a smaller upper section adjacent to a lower section.

60. The balloon catheter of claim 53 wherein an opening in the distal engaging mechanism has a keyhole shape with an upper section adjacent to a smaller lower section.

61. The balloon catheter of claim 56 wherein an opening in the proximal engaging mechanism has a keyhole shape with a smaller upper section adjacent to a lower section.

62. The balloon catheter of claim 56 wherein an opening in the proximal engaging mechanism has a keyhole shape with an upper section adjacent to a smaller lower section.

63. The balloon catheter of claim 53 wherein the inflation lumen is located on the outside surface of the balloon catheter.

64. The balloon catheter of claim 53 wherein the inflation lumen is located inside of the balloon catheter.

65. A balloon catheter comprising:
a proximal end, a distal end and a longitudinal axis therebetween;
an inflation lumen located along the longitudinal axis of the balloon catheter;
a balloon supported by the balloon catheter, an inner surface of the balloon in communication with the inflation lumen; and
a channel along an outside surface of the balloon catheter.

66. The balloon catheter of claim 65 further comprising a lumen extending from the channel to the proximal end of the balloon catheter.

67. The balloon catheter of claim 65 further comprising a distal channel engaging support located along the distal end of the balloon catheter.

68. The balloon catheter of claim 65 further comprising a proximal channel engaging support located proximal to the distal end of the balloon catheter.

69. The balloon catheter of claim 65 further comprising a distal channel engaging support at the distal end of the balloon catheter and a proximal channel engaging support located proximal to the distal channel engaging support.

70. The balloon catheter of claim 65 wherein the inflation lumen is located on the outside surface of the balloon catheter.

71. The balloon catheter of claim 65 wherein the inflation lumen is located inside of the balloon catheter.

72. The balloon catheter of claim 65 wherein the balloon is non-compliant.

73. The balloon catheter of claim 65 wherein an injection of a medium through the inflation lumen expands the balloon.

74. The balloon catheter of claim 65 wherein the balloon is located over a portion of a circumference of the balloon catheter.

75. An ultrasonic probe comprising:
a proximal end, a distal end and a longitudinal axis therebetween;
a proximal section located proximal to the distal end; and
a flexible section located between the distal end and the proximal section, wherein the flexible section comprises a diameter smaller than both a diameter of the proximal section of the ultrasonic probe and a diameter of the distal end of the ultrasonic probe.

76. The ultrasonic probe of claim 75 wherein the flexible section comprises a flexibility that allows the ultrasonic probe to deflect without affecting the mechanical or ultrasonic properties of the ultrasonic probe.

77. The ultrasonic probe of claim 75 wherein the small diameter flexible section allows greater flexibility for bending the ultrasonic probe.

78. The ultrasonic probe of claim 75 wherein a diameter of the ultrasonic probe decreases from the proximal section to the flexible section over a diameter transition.

79. The ultrasonic probe of claim 75 wherein a diameter of the ultrasonic probe increases from the flexible section to the distal end over a diameter transition.

80. The ultrasonic probe of claim 75 wherein a diameter of the ultrasonic probe gradually tapers from a larger diameter of the proximal section of the ultrasonic probe to a smaller diameter of the flexible section of the ultrasonic probe.

81. The ultrasonic probe of claim 75 wherein a diameter of the ultrasonic probe gradually tapers from a smaller diameter of the flexible section of the ultrasonic probe to a larger diameter of the distal end of the ultrasonic probe.

82. The ultrasonic probe of claim 75 wherein a plurality of transverse nodes and a plurality of transverse anti-nodes caused by a transverse ultrasonic vibration of the ultrasonic probe are located along the flexible section, the proximal section and the distal end of the ultrasonic probe.
